(19) United States
(12) Patent Application Publication
(10) Pub. No.: US 2024/0218458 A1
(43) Pub. Date: Jul. 4, 2024

(54) A MULTIPLEX CRISPR BASED ASSAY FOR THE SIMULTANEOUS DETECTION OF CHLAMYDIA TRACHOMATIS AND NEISSERIA GONORRHOEAE

(71) Applicants: University of Maryland, Baltimore, Baltimore, MD (US); University of Maryland, Baltimore County, Baltimore, MD (US); The Penn State Research Foundation, University Park, PA (US)

(72) Inventors: Dipanjan Pan, State College, PA (US); Parikshit Moitra, State College, PA (US); Ketan Dighe, Ellicott City, MD (US)

(21) Appl. No.: 18/332,359

(22) Filed: Jun. 9, 2023

Related U.S. Application Data
(60) Provisional application No. 63/350,586, filed on Jun. 9, 2022.

(51) Int. Cl. C12Q 1/689; C12N 9/22; C12N 15/11

(52) U.S. Cl. CPC C12Q 1/689; C12N 9/22; C12N 15/11; C12N 2310/20

(57) ABSTRACT

The present invention provides methods of detecting the presence or absence of nucleic acid from Chlamydia trachomatis and the presence or absence of nucleic acid from Neisseria gonorrhoeae in a sample from a subject using CRISPR nucleases and crRNAs specific for Chlamydia trachomatis and Neisseria gonorrhoeae nucleic acid, kits for carrying out the methods and methods of treating subjects infected with Chlamydia trachomatis and/or Neisseria gonorrhoeae.

### CROSS-REFERENCE TO RELATED APPLICATIONS

This application claims the benefit of U.S. Provisional Appl. No. 63/350,586, filed on Jun. 9, 2022, the contents of which are hereby incorporated by reference in their entirety.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 39,886 Byte (xml) file named “Sequence_listing.xml,” created on Mar. 20, 2024.

### FIELD OF THE INVENTION

The field of the invention relates to infectious diseases, in particular sexually transmitted infections and methods of their detection and treatment.

### BACKGROUND OF THE INVENTION

Sexually transmitted infections (STI) are one of the most common and widespread diseases around the world (Wiesenfeld, H. C., New England Journal of Medicine, (2017), 376: 765-773; Taylor, B. D., Infection and Drug Resistance, (2011), 19). Among them, infections caused by Chlamydia trachomatis (CT) and Neisseria gonorrhoeae (NG) stand out as the first and second most reported bacterial infections, with approximately 1.9 million and 620 K reported cases, respectively in the United States alone in 2019 (Chlamydia—STD information from CDC—www.cdc.gov/std/chlamydia/default.htm (accessed 2022 Oct. 2); Report on global sexually transmitted infection surveillance 2018 https://www.who.int/publications/i/item/9789241565691 (accessed 2022 Oct. 3); Rowley et al., 2016. Bulletin of the World Health Organization, (2019), 97:548-562P). That makes these STIs an epidemic and can lead to numerous economic and health consequences (Wynn et al., Sexually Transmitted Diseases, (2019), 47:5-11; Newman et al., PLOS ONE 2015, 10:e0143304; Soler et al., Biosensors and Bioelectronics, (2017), 94:560-567). While Chlamydia caused by Chlamydia trachomatis is usually asymptomatic, particularly in women, it can contribute to the development of pelvic inflammatory disease (PID), leading to infertility, pregnancy complications such as ectopic pregnancy, and chronic pelvic pain. In men, this can lead to scarring of the urogenital system causing urination problems, sterility, and painful inflammation of the epididymis (Gaydos et al., Expert Review of Anti-infective Therapy, (2014), 12:657-672; Hoover et al., Obstetrics & Gynecology, (2010), 115:495-502; Tao et al., Southern Medical Journal, (2017), 110:18-24; Hoover et al., Obstetrics & Gynecology, (2010), 115:495-502; Fung et al., Sexually Transmitted Infections, (2007), 83:304-309). Furthermore, Chlamydia infection can facilitate the transmission of the human immunodeficiency virus (HIV) and be transmitted vertically to infants, leading to neonatal infections, such as ophthalmia neonatorum and pneumonia (Chlamydia—STD information from CDC—www.cdc.gov/std/chlamydia/default.htm (accessed 2022 Oct. 2). In 2016, over 1.5 million cases of this infection were reported, corresponding to a rate of nearly 500 cases per 100,000 population (Wiesenfeld, H. C., New England Journal of Medicine, (2017), 376: 765-773; Taylor, B. D., Infection and Drug Resistance, (2011), 19; Chlamydia—STD information from CDC—www.cdc.gov/std/chlamydia/default.htm (accessed 2022 Oct. 2; Report on global sexually transmitted infection surveillance 2018 https://www.who.int/publications/i/item/9789241565691 (accessed 2022 Oct. 3); Rowley et al., 2016. Bulletin of the World Health Organization, (2019), 97:548-562P; Wynn et al., Sexually Transmitted Diseases, (2019), 47:5-11; Newman et al., PLOS ONE 2015, 10:e0143304). Rates of reported cases have increased over the few years since, though this may at least partially be due to increased screening as well as improved diagnosis and reporting. Like Chlamydia, gonorrhea-caused by Neisseria gonorrhoeae can also contribute to the development of pelvic inflammatory disease (PID) and its complications, as well as the transmission of HIV (Wiesenfeld, H. C., New England Journal of Medicine, (2017), 376: 765-773; Taylor, B. D., Infection and Drug Resistance, (2011), 19; Chlamydia—STD information from CDC—www.cdc.gov/std/chlamydia/default.htm (accessed 2022 Oct. 2; Report on global sexually transmitted infection surveillance 2018 https://www.who.int/publications/i/item/9789241565691 (accessed 2022 Oct. 3); Rowley et al., 2016. Bulletin of the World Health Organization, (2019), 97:548-562P; Wynn et al., Sexually Transmitted Diseases, (2019), 47:5-11; Newman et al., PLOS ONE 2015, 10:e0143304; Soler et al., Biosensors and Bioelectronics, (2017), 94:560-567; Hillis et al., New England Journal of Medicine, (1996), 334:1399-1401; Scholes et al., New England Journal of Medicine, (1996), 334:1362-1366; Recommendations for the Laboratory-Based Detection of Chlamydia trachomatis and Neisseria gonorrhoeae 2014 https://www.cdc.gov/mmwr/preview/mmwrhtml/rr6302a1.htm (accessed 2022 Oct. 3; Gaydos et al., Expert Review of Anti-infective Therapy, (2014), 12:657-672; Hoover et al., Obstetrics & Gynecology, (2010), 115:495-502; Tao et al., Southern Medical Journal, (2017), 110:18-24; Hoover et al., Obstetrics & Gynecology, (2010), 115:495-502; Fung et al., Sexually Transmitted Infections, (2007), 83:304-309; Hosenfeld et al., Sexually Transmitted Diseases, (2009), 36:478-489; Workowski et al., MMWR. Recommendations and Reports, (2021), 70:1-187). N. gonorrhoeae has progressively developed resistance to each of the antibiotics used to treat gonorrhea, rendering surveillance of antibiotic susceptibility increasingly important (Knapp, J., et al., Emerging Infectious Diseases, (1997), 3:33-39; Whittington et al., Sexually Transmitted Diseases, (1988), 15:202-210; Ison et al., Sexually Transmitted Infections, (1996), 72: 253-257). In 2016, nearly 500,000 cases of this infection were reported, corresponding to a rate of almost 150 cases per 100,000 population. Rates of reported cases have increased nearly 50 percent in the last decade after reaching a historic low in 2009, though again, this may in part be due to increased screening as well as improved diagnosis and reporting (Chlamydia—STD information from CDC—www.cdc.gov/std/chlamydia/default.htm (accessed 2022 Oct. 2); Report on global sexually transmitted infection surveillance 2018 https://www.who.int/publications/i/item/9789241565691 (accessed 2022 Oct. 3); Rowley et al., 2016. Bulletin of the World Health Organization, (2019), 97:548-562P). Therefore, the increasing rates of Chlamydia and gonorrhea-like STIs pose a global public health issue.

The prevalence of these two infections is such that organizations like the Centers for Disease Control and Prevention (CDC), United States Preventive Services Task Force (USPSTF), American Academy of Family Physicians (AAFP), and American College of Obstetricians and Gynecologists (ACOG) recommended that women should be screened regularly for these infections, not only for their own health but because STIs can cause congenital abnormalities and death in unborn babies ((Chlamydia—STD information from CDC—www.cdc.gov/std/chlamydia/default.htm (accessed 2022 Oct. 2); Report on global sexually transmitted infection surveillance 2018 https://www.who.int/publications/i/item/9789241565691 (accessed 2022 Oct. 3); Rowley et al., 2016. Bulletin of the World Health Organization, (2019), 97:548-562P; Screening for Chlamydial Infection: U.S. Preventive Services Task Force Recommendation Statement. Annals of Internal Medicine, (2007), 147:128; LeFevre et al., Annals of Internal Medicine, (2014), 161:902). Patients who test positive for these infections often exhibit common symptoms or show no symptoms at all, making them so insidious. Furthermore, both of these infections tend to become antibiotic-resistant over time. STIs are typically treated presumptively due to the lack of accurate tests or delayed results, resulting in over- and undertreatment. At the ED settings, empiric treatment decisions are made leading to overtreatment of ˜30% of the population with no STIs and the undertreatment of ˜40% of patients with confirmed CT & NG cases. In some studies of individual EDs, overtreatment rates are greater than 80% and undertreatment greater than 50%.²⁵⁻²⁷ If left untreated, cases may result in long-term morbidity, multiple medical complications, and adverse consequences. Additionally, unnecessarily using antibiotics can cause adverse effects and increase antibiotic resistance (Holley et al., The American Journal of Emergency Medicine, (2015), 33:1265-1268; Anaene et al., International Journal of Infectious Diseases, (2016), 53:34-38; Pearson et al., Emerg Infect Dis, (2017), 23:367-369). To ensure timely infection control measures in the ED setting and to be able to use antibiotics appropriately, rapid and accurate identification of CT & NG is essential.

Additionally, it is also important to identify these infections before certain procedures like the insertion of an Intrauterine Device (IUD). If an IUD is inserted and the STI is present at the time of insertion but is not caught, the woman is at high risk for developing PID and its associated complications (Jatlaoui et al., Contraception, (2016), 94:701-712; El Ayadi et al., Journal of Pediatric and Adolescent Gynecology, (2021), 34:355-361; Grentzer et al., Contraception, (2014), 90:292). As a result, a patient must make a return appointment at their doctor's office to receive the treatment, which also requires compliance, visit costs/insurance coverage, reliable transportation, and time off work. Alternatively, patients must make an appointment for testing prior to the implantation resulting in similar burdens on time, insurance, and transportation (Uscher-Pines et at, Am J Manag Care, (2013), 19:47-59). In the absence of these steps, the patient may not receive proper treatment and risks spreading the disease to others, as well as adverse health consequences for the individual and, if pregnant, the unborn child (Grentzer et al, Contraception, (2014), 90:292). The current test entails a Nucleic Acid Amplification Test (NAAT) and takes approximately 2-3 days for the results. For men, it requires a urine sample, while for women it requires a cervical swab (Gaydos et al., Sex Transm Dis, (2008), 35:S45-S50; Land et al., Nature Microbiology, (2018), 4:46-54). In addition to being harmful to the patient's health and finances, this can pose a threat to the community as well. In order to treat the patients properly, and eliminate the risk of under- or overtreatment probabilities, a fast diagnostic assay for CT & NG is an urgent unmet clinical need of modern society.

The testing for CT & NG must consider several factors in selecting the appropriate diagnostic modality, including accuracy, cost, ease, turnaround time, and invasiveness (Land et al., Nature Microbiology, (2018), 4:46-54). Historically, bacterial culture was regarded as the gold standard for testing Chlamydia and gonorrhea (Papp et al, Infectious Diseases in Obstetrics and Gynecology, (2016), 2016:1-17). The major challenge with this modality is maintaining the viability of organisms during transportation and storage; additionally, culture methods for isolating CT are difficult to standardize, technically difficult, expensive, and insensitive. For CT & NG, non-culture modalities were developed, including enzyme immunoassays (EIAs) and direct fluorescent antibody (DFA) tests (Herbst de Cortina et al, Infectious Diseases in Obstetrics and Gynecology, (2016), 1-17). These tests, however, still face technical difficulties, are expensive, and are insensitive. Next, nucleic acid hybridization tests were introduced; these detect either CT or NG specific deoxyribonucleic acid (DNA) or ribonucleic acid (RNA) sequences (Martin et al., Journal of Clinical Microbiology, (2004). 42:4749-4758; Garrett et al., BMJ Open, (2019), 9:e026888; Harding-Esch e; al, EBioMedicine, (2018), 28:120-127). While these tests are more convenient and in remote settings more reliable than their culture predecessor, they are still insensitive, particularly for CT. The subsequent development of nucleic acid amplification tests (NAATs), which amplify and detect CT & NG—specific DNA or RNA sequences, led to a convenient, reliable, and sensitive option for the diagnosis of Chlamydia and gonorrhea.

NAATs, including polymerase chain reaction (PCR), transcription-mediated amplification (TMA), and strand displacement amplification (SDA), are now considered the gold standard for diagnosing Chlamydia (Martin et al., Journal of Clinical Microbiology, (2004), 42:4749-4758; Garrett et al, BMJ Open, (2019), 9:e026888; Harding-Esch et al, EBioMedicine, (2018), 28:120-127. As with Chlamydia, NAATs are now considered the gold standard for diagnosing gonorrhea as well. Although established genomic diagnostic tools, such as polymerase chain reaction (PCR), have superior specificity and sensitivity, they have several drawbacks in terms of usability and costs. Furthermore, most of these established nucleic acid sensing methods require complicated, bulky equipment and trained personnel. To address this issue many companies have started to offer modular platforms so the NAAT assay can be performed directly on specimens collected from patients without the need for manual or off-board specimen preparation. Such technologies include the Cepheid Xpert® CT/NG assay, binx health io CT/NG Assay, Truelab Real Time micro-PCR system (Molbio Diagnostics Pvt Ltd), and STI Array (Randox Biosciences) (Garrett et al., BMJ Open, (2019), 9:e026888; Harding-Esch et al., EBioMedicine, (2018), 28:120-127; Van Der Pol et al., Expert Review of Molecular Diagnostics, (2021), 21:861-868; Adamson et al., Archives of Pathology & Laboratory Medicine, (2020). 144:1344-1351; Shin et al., Scientific Reports, (2017), 7 (1); Doernberg et al., Clinical Infectious Diseases, 2019). The listed technologies can provide results in approximately 30-90 minutes with a low false positive rate as demonstrated in independent studies. However, these tests require access to company specific instruments such as the GeneXpert system, of which only 5,000 are available in the US. Apart from amplification and detection, the tests also require DNA extraction as a separate step, which is a key constraint on scalability and could pose a major hurdle for testing in a physician's office. Newer isothermal amplification methods (e.g., LAMP) are faster, but their sensitivity and specificity are compromised (Chen et al., Frontiers in Microbiology, (2022), 13). The Abbott ID NOW isothermal amplification technology claims the delivery of positive results in less than 30 minutes while offering a device of portable size and weight. However, this test also requires specialized instrumentation and has limited availability as well as recent reports of possible inaccuracy.

In addition to the NAAT-based POC tests for C trachomatis mentioned above, there are also several POC tests that can detect C. trachomatis and N. gonorrhoeae antigen Although many of the available lateral flow assay tests for C. trachomatis and N. gonorrhoeae show promise, they generally underperform compared with NAAT-based POC tests and are not suitable for use in screening populations in a physician's office. There is an ongoing need for better-performing assays and more rigorous evaluations to inform their potential use.

Therefore, nucleic acid based diagnostic tools are needed, which combine the ease of use, cost-effectiveness, and speed of isothermal amplification with high sensitivity and specificity.

### SUMMARY OF THE INVENTION

It is to be understood that both the foregoing general description of the embodiments and the following detailed description are exemplary, and thus do not restrict the scope of the embodiments.

In one aspect, the invention provides a method of detecting the presence or absence of nucleic acid from Chlamydia trachomatis and the presence or absence of nucleic acid from Neisseria gonorrhoeae in a sample from a subject, comprising
a. providing the sample from the subject;
b. contacting the sample with i) a complex comprising a CRISPR nuclease having collateral nuclease activity and a crRNA comprising a nucleic acid sequence that is complementary to a nucleic acid sequence of Chlamydia trachomatis; and ii) a first single stranded nucleic acid linker probe;
c. contacting the sample with iii) a complex comprising a CRISPR nuclease having collateral nuclease activity and a crRNA comprising a nucleic acid sequence that is complementary to a nucleic acid sequence of Neisseria gonorrhoeae; and iv) a second single stranded nucleic acid linker probe;
d. incubating the sample with the complexes of parts i) and iii) and single stranded linker probes of parts ii) and iv) for a period of time;
   wherein when the nucleic acid sequence of Chlamydia trachomatis is present in the sample, the collateral nuclease activity of the complex of part i) is activated and the first single stranded nucleic acid linker probe or part ii) is cleaved by the nuclease;
   wherein when the nucleic acid sequence of Chlamydia trachomatis is not present in the sample, the collateral nuclease activity of the complex of part i) is not activated and the first single stranded nucleic acid linker probe of part ii) is not cleaved;
   wherein when the nucleic acid sequence of Neisseria gonorrhoeae is present in the sample, the collateral nuclease activity of the complex of part iii) is activated and the second single stranded nucleic acid linker probe of part iv) is cleaved by the nuclease;
   wherein when the nucleic acid sequence of Neisseria gonorrhoeae is not present in the sample, the collateral nuclease activity of the complex of part iii) is not activated and the second single stranded nucleic acid linker probe of part iv) is not cleaved;
e. contacting the sample with a first single stranded nucleic acid that hybridizes to a first nucleic acid sequence of the first single stranded nucleic acid linker probe of part ii) and a second single stranded nucleic acid that hybridizes to a second nucleic acid sequence of the first single stranded nucleic acid linker probe of part ii);
   wherein the first and second single stranded nucleic acids hybridize to the first single stranded nucleic acid linker probe of part ii) when the first single stranded nucleic acid linker probe of part ii) is not cleaved, to form a complex comprising the first single stranded nucleic acid linker probe of part ii) and the first and second single stranded nucleic acids;
f. contacting the sample with a third single stranded nucleic acid that hybridizes to a first nucleic acid sequence of the second single stranded nucleic acid linker probe of part iv) and a fourth single stranded nucleic acid that hybridizes to a second nucleic acid sequence of the second single stranded nucleic acid linker probe of part iv);
   wherein the third and fourth single stranded nucleic acids hybridize to the second single stranded nucleic acid linker probe of part iv) when the second single stranded nucleic acid linker probe of part iv) is not cleaved, to form a complex comprising the second single stranded nucleic acid linker probe of part iv) and the third and fourth single stranded nucleic acids;
g. detecting the presence or absence of the complex comprising the first single stranded nucleic acid linker probe of part ii) hybridized to the first and second single stranded nucleic acids, thereby detecting the presence or absence of nucleic acid from Chlamydia trachomatis in the sample; and
h. detecting the presence or absence of the complex comprising the second single stranded nucleic acid linker probe of part iv) hybridized to the third and fourth single stranded nucleic acids, thereby detecting the presence or absence of nucleic acid from Neisseria gonorrhoeae in the sample.

In another aspect, the invention provides a kit, comprising
a. one or more test strips, one or more solid supports, one or more beads, one or more flow paths in a lateral flow immunoassay device, and/or one or more wells in a microtiter plate;
b. a CRISPR nuclease having collateral nuclease activity;
c. a crRNA comprising a nucleic acid sequence that is complementary to a nucleic acid sequence of Chlamydia trachomatis, a first single stranded nucleic acid linker probe, a first single stranded nucleic acid that is complementary to a first nucleic acid sequence of the first single stranded nucleic acid linker probe and a second single stranded nucleic acid that is complementary to a second nucleic acid sequence of the first single stranded nucleic acid linker probe;
d. a crRNA comprising a nucleic acid sequence that is complementary to a nucleic acid sequence of Neisseria gonorrhoeae, a second single stranded nucleic acid linker probe, a first single stranded nucleic acid that is complementary to a first nucleic acid sequence of the second single stranded nucleic acid linker probe and a second single stranded nucleic acid that is complementary to a second nucleic acid sequence of the second single stranded nucleic acid linker probe;
e. optionally one or more solutions suitable for use; and
f. optionally instructions for use.

In another aspect, the invention provides a method of detecting the presence or absence of nucleic acid from Chlamydia trachomatis in a sample from a subject, comprising
a. providing the sample from the subject;
b. contacting the sample with i) a complex comprising a CRISPR nuclease having collateral nuclease activity and a crRNA comprising a nucleic acid sequence that is complementary to a nucleic acid sequence of Chlamydia trachomatis; and ii) a single stranded nucleic acid linker probe;
c. incubating the sample with the complex of part i) and single stranded linker probe of part ii) for a period of time;
   wherein when the nucleic acid sequence of Chlamydia trachomatis is present in the sample, the collateral nuclease activity of the complex of part i) is activated and the single stranded nucleic acid linker probe or part ii) is cleaved by the nuclease;
   wherein when the nucleic acid sequence of Chlamydia trachomatis is not present in the sample, the collateral nuclease activity of the complex of part i) is not activated and the single stranded nucleic acid linker probe of part ii) is not cleaved;
d. contacting the sample with a first single stranded nucleic acid that hybridizes to a first nucleic acid sequence of the single stranded nucleic acid linker probe of part ii) and a second single stranded nucleic acid that hybridizes to a second nucleic acid sequence of the single stranded nucleic acid linker probe of part ii);
   wherein the first and second single stranded nucleic acids hybridize to the single stranded nucleic acid linker probe of part ii) when the single stranded nucleic acid linker probe of part ii) is not cleaved, to form a complex comprising the single stranded nucleic acid linker probe of part ii) and the first and second single stranded nucleic acids;
e. detecting the presence or absence of the complex comprising the single stranded nucleic acid linker probe of part ii) hybridized to the first and second single stranded nucleic acids, thereby detecting the presence or absence of nucleic acid from Chlamydia trachomatis in the sample.

In another aspect, the invention provides a kit, comprising
a. one or more test strips, one or more solid supports, one or more beads, one or more flow paths in a lateral flow immunoassay device, and/or one or more wells in a microtiter plate;
b. a CRISPR nuclease having collateral nuclease activity;
c. a crRNA comprising a nucleic acid sequence that is complementary to a nucleic acid sequence of Chlamydia trachomatis, a single stranded nucleic acid linker probe, a first single stranded nucleic acid that is complementary to a first nucleic acid sequence of the single stranded nucleic acid linker probe and a second single stranded nucleic acid that is complementary to a second nucleic acid sequence of the single stranded nucleic acid linker probe;
d. optionally one or more solutions suitable for use; and
e. optionally instructions for use.

In another aspect, the invention provides a method of detecting the presence or absence of nucleic acid from Neisseria gonorrhoeae in a sample from a subject, comprising
a. providing the sample from the subject;
b. contacting the sample with i) a complex comprising a CRISPR nuclease having collateral nuclease activity and a crRNA comprising a nucleic acid sequence that is complementary to a nucleic acid sequence of Neisseria gonorrhoeae; and ii) a single stranded nucleic acid linker probe;
c. incubating the sample with the complexes of part i) and single stranded linker probe of part ii) for a period of time;
wherein when the nucleic acid sequence of Neisseria gonorrhoeae is present in the sample, the collateral nuclease activity of the complex of part i) is activated and the single stranded nucleic acid linker probe of part ii) is cleaved by the nuclease;
wherein when the nucleic acid sequence of Neisseria gonorrhoeae is not present in the sample, the collateral nuclease activity of the complex of part i) is not activated and the single stranded nucleic acid linker probe of part ii) is not cleaved;
d. contacting the sample with a first single stranded nucleic acid that hybridizes to a first nucleic acid sequence of the single stranded nucleic acid linker probe of part ii) and a second single stranded nucleic acid that hybridizes to a second nucleic acid sequence of the single stranded nucleic acid linker probe of part ii);
wherein the first and second single stranded nucleic acids hybridize to the single stranded nucleic acid linker probe of part ii) when the single stranded nucleic acid linker probe of part ii) is not cleaved, to form a complex comprising the single stranded nucleic acid linker probe of part ii) and the first and second single stranded nucleic acids;
e. detecting the presence or absence of the complex comprising the stranded nucleic acid linker probe of part ii) hybridized to the first and second single stranded nucleic acids, thereby detecting the presence or absence of nucleic acid from Neisseria gonorrhoeae in the sample.

In another aspect, the invention provides a kit, comprising
a. one or more test strips, one or more solid supports, one or more beads, one or more flow paths in a lateral flow immunoassay device, and/or one or more wells in a microtiter plate;
b. a CRISPR nuclease having collateral nuclease activity;
c. a crRNA comprising a nucleic acid sequence that is complementary to a nucleic acid sequence of Neisseria gonorrhoeae, a single stranded nucleic acid linker probe, a first single stranded nucleic acid that is complementary to a first nucleic acid sequence of the single stranded nucleic acid linker probe and a second single stranded nucleic acid that is complementary to a second nucleic acid sequence of the single stranded nucleic acid linker probe;
d. optionally one or more solutions suitable for use; and
e. optionally instructions for use.

In some embodiments, the methods further comprise administering to the subject an effective amount of one or more agents to treat an infection caused by Chlamydia trachomatis and/or Neisseria gonorrhoeae.

In some embodiments, when nucleic acid from Chlamydia trachomatis is present in the subject's sample, the one or more agents is selected from the group consisting of doxycycline, azithromycin, erythromycin, levofloxacin, and amoxicillin.

In some embodiments, when nucleic acid from Neisseria gonorrhoeae is present in the subject's sample, the one or more agents is selected from the group consisting of ceftriaxone, cefotaxime, ceftizoxime, ceftriaxone, gentamicin and cefixime.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Concept diagram and process flow for the CRISPR-Cas12 directed nucleic acid (CDNA) based lateral flow assay for the selective detection of CT and NG with no additional amplification steps.

FIG. 2. (a) Schematic representation of agglomeration induced CRISPR directed selective detection of CT and NG; (b) Designated crRNA, linker probe and ssDNA sequences with their target binding and binding disruption energies; Change in absorbance of suitably designed AuNPs added with crRNA and ssDNAs targeted for (c) CT and (f) NG: DNA extracted from 1—CT +ve, 2—Both CT and NG +ve, 3—NG +ve and 4—Both CT and NG −ve samples; Change in morphology of suitably designed AuNPs added with crRNA and ssDNAs targeted for (d, e) CT and (g, h) NG: (d) CT +ve, (e) CT −ve, (g) NG +ve and (h) NG −ve samples.

FIG. 3. Performance of the CDNA assay in the form of confusion matrix towards the selective diagnosis of (a) CT and (c) NG; The respective sensing parameters are highlighted in (b) and (d) for CT and NG assay respectively.

FIG. 4. Performance of the CDNA assay in the form of confusion matrix towards the selective diagnosis of (a) CT and (b) NG directly from the clinical samples without any additional nucleic acid isolation, purification, and amplification steps.

FIG. 5. Schematic representation of operating principle of the proposed dipstick assay. In the presence of the target bacterial DNA in the suspension, the linker probe gets chopped off, while in absence of target DNA, the linker probe remains intact. The labeled ssDNAs will then bind with their complementary linker probe sequence depending on the presence/absence of the target DNA.

FIG. 6. Feasibility of the concept with CT/NG positive (N=2) and negative (N=2) samples on a dipstick based lateral flow platform.

FIG. 7. Schematic of a CRISPR CTNG detection assay.

FIG. 8. CRISPR-CT detection results.

FIG. 9. CT-linker probe concentration optimization results.

FIG. 10. CT (Au-ASO) optimization results.

FIG. 11. Analysis of CRISPR-CT clinical samples. 60 Clinical samples were analyzed with the following optimized conditions: linker probe conc.—3.1 μM; Au-ASO conc.—3× dilution; Cr-RNA conc.—30 nM; Cas12a conc.—30 nM; sample DNA conc.—˜30 nM; incubation temp—37° C.

FIG. 12. CRISPR-NG detection results.

FIG. 14. NG-linker probe concentration optimization results.

FIG. 14. NG (Au-ASO) optimization results.

FIG. 15. Analysis of CRISPR-NG clinical samples. 60 Clinical samples were analyzed with the following optimized conditions: linker probe conc.—3.1 μM; Au-ASO conc.—2× dilution; Cr-RNA conc.—30 nM; Cas12a conc.—30 nM; sample DNA conc.—˜30 nM; incubation temp—37° C.

FIG. 16. Analysis of CRISPR-NG direct clinical samples. 40 Clinical samples were analyzed with the following optimized conditions: linker probe conc.—3.1 μM; Au-ASO conc.—2× dilution; Cr-RNA conc.—30 nM; Cas12a conc.—30 nM; sample DNA conc.—˜30 nM; incubation temp—37° C.

FIG. 17. Analysis of CRISPR-CT direct clinical samples. 40 Clinical samples were analyzed with the following optimized conditions: linker probe conc.—3.1 μM; Au-ASO conc.—2× dilution; Cr-RNA conc.—30 nM; Cas12a conc.—30 nM; sample DNA conc.—˜30 nM; incubation temp—37° C.

### DETAILED DESCRIPTION OF THE INVENTION

The present invention provides a Clustered Regularly Interspaced Short Palindromic Repeats (CRISPR)-directed nucleic acid detection assay for detection of Chlamydia trachomatis and Neisseria gonorrhoeae infections. The present methods overcome the hurdles preventing rapid, accurate diagnostics. The present methods are simple, fast, and are highly portable genomic diagnostic tools that can be performed at the point of care, even in resource-limited settings without laboratory access. Test results can be interpreted using dipstick assays (or Lateral Flow Assays (LFAs)) rather than sophisticated equipment as often found in CLIA-waived platforms. Each assay can include an internal positive control sensitive to inhibitors. Using CRISPR-associated nuclease (Cas)-based approaches and collateral cleavage activity by effector proteins such as Cas12, the present inventors developed an alternative to creating genomic diagnostic tools that meet the point of care sensing specifications.

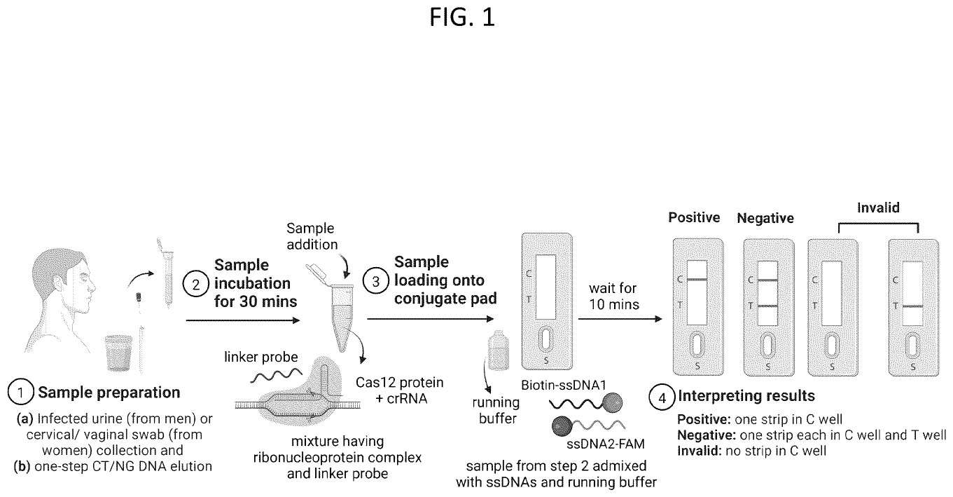

As shown herein, when these effector proteins are associated with a CRISPR RNA (crRNA), they can specifically complement with a target sequence from Chlamydia trachomatis or Neisseria gonorrhoeae, inducing enzymatic cleavage of both the targeted sequence and nontargeted collateral cleavage of all nucleic acids (e.g., ssDNA) in the environment. Furthermore, a dipstick assay can be used for the rapid (e.g., <40 min), easy-to-implement and accurate multiplexed diagnosis of Chlamydia trachomatis and Neisseria gonorrhoeae directly from the clinical samples (FIG. 1) collected during the office visits.

The present invention provides crRNAs targeted towards the genes of Chlamydia trachomatis and Neisseria gonorrhoea, respectively. For Chlamydia trachomatis, cryptic plasmid ORF6 segment is targeted, while for Neisseria gonorrhoea, major outer membrane protein, porB is targeted, for the generation of crRNA. An infectious cervical swab (for women) and urine (for men) sample can be collected, and bacterial DNA can be isolated by a standardized one-step DNA elution protocol. The sample can then be incubated with a mixture of ribonucleoprotein (RNP) complex (e.g., mixture of Cas12 protein and crRNA) and a suitably designed linker probe (ssDNA) for 30 mins at room temperature. In the presence of the target CT/NG DNA, the RNP complex turns its collateral cleavage activity on and cleaves the linker probe present in the suspension. The suspension can then be admixed with a mixture of newly designed single-stranded DNAs (ssDNAs), e.g., labeled with 6-carboxyfluorescein (FAM) and biotin respectively, mixed with running buffer and incubated for 10 mins. In the presence of the target bacterial DNA, the linker probe will be cleaved by the Cas enzyme and the labeled ssDNAs cannot agglomerate and do not show up on the test line of the dipstick. However, in absence of the target DNA, the labeled ssDNAs bind with their complementary linker probe sequence to demonstrate a prominent test line on the dipstick (FIG. 1). Results provided herein demonstrated validation of the methods on de-identified clinical cervical swab and urine samples. The methods provide a visual and faster alternative to RT-PCR assays that provide 99% positive and negative predictive agreements without the need for additional nucleic acid amplification steps.

Despite the high risk for transmission and complications, a significant portion of patients at STI clinics fail to follow up. In addition, clinics and doctors find laboratory testing for infectious diseases to be costly and time-consuming. Testing delays are caused by the transportation of samples to central laboratories, which increases the overall cost of testing. The present invention solves these shortcoming with a high-accuracy, pregnancy-style multiplexed test for Chlamydia trachomatis and Neisseria gonorrhoeae that can impact how doctors diagnose and treat millions of infected patients each year. By allowing patients to be tested and treated during a single clinical visit, this molecular diagnostic technology can reduce the spread and pain of STI infections. As a result of the test, patient management will be improved, clinic costs will be reduced, and public health will be improved overall. With the implementation of these new methods, it is expected that overtreatment and undertreatment cases will also be reduced, which will help to prevent antibiotic resistance in these bacteria.

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

The terms “nucleic acid,” and “polynucleotide,” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids. The term “sequence” relates to a nucleotide sequence of any length, which can be DNA or RNA; can be linear, circular or branched and can be either single-stranded or double stranded.

The term “identity” relates to an exact nucleotide-to-nucleotide or amino acid-to-amino acid correspondence of two polynucleotides or polypeptide sequences, respectively. Two or more sequences (polynucleotide or amino acid) can be compared by determining their percent identity. Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

“Sequence similarity” between polynucleotides can be determined by hybridization of polynucleotides under conditions that allow formation of stable duplexes between homologous regions, followed by digestion with single-stranded-specific nuclease(s), and size determination of the digested fragments.

“Subject,” as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In one embodiment, the subject is a human. In some embodiments, the subject is a mouse.

In one embodiment, the invention provides a method of detecting the presence or absence of nucleic acid from Chlamydia trachomatis and the presence or absence of nucleic acid from Neisseria gonorrhoeae in a sample from a subject, comprising
a. providing the sample from the subject;
b. contacting the sample with i) a complex comprising a CRISPR nuclease having collateral nuclease activity and a crRNA comprising a nucleic acid sequence that is complementary to a nucleic acid sequence of Chlamydia trachomatis; and ii) a first single stranded nucleic acid linker probe;
c. contacting the sample with iii) a complex comprising a CRISPR nuclease having collateral nuclease activity and a crRNA comprising a nucleic acid sequence that is complementary to a nucleic acid sequence of Neisseria gonorrhoeae; and iv) a second single stranded nucleic acid linker probe;
d. incubating the sample with the complexes of parts i) and iii) and single stranded linker probes of parts ii) and iv) for a period of time;
   wherein when the nucleic acid sequence of Chlamydia trachomatis is present in the sample, the collateral nuclease activity of the complex of part i) is activated and the first single stranded nucleic acid linker probe or part ii) is cleaved by the nuclease;
   wherein when the nucleic acid sequence of Chlamydia trachomatis is not present in the sample, the collateral nuclease activity of the complex of part i) is not activated and the first single stranded nucleic acid linker probe of part ii) is not cleaved;
   wherein when the nucleic acid sequence of Neisseria gonorrhoeae is present in the sample, the collateral nuclease activity of the complex of part iii) is activated and the second single stranded nucleic acid linker probe of part iv) is cleaved by the nuclease;
   wherein when the nucleic acid sequence of Neisseria gonorrhoeae is not present in the sample, the collateral nuclease activity of the complex of part iii) is not activated and the second single stranded nucleic acid linker probe of part iv) is not cleaved;
e. contacting the sample with a first single stranded nucleic acid that hybridizes to a first nucleic acid sequence of the first single stranded nucleic acid linker probe of part ii) and a second single stranded nucleic acid that hybridizes to a second nucleic acid sequence of the first single stranded nucleic acid linker probe of part ii);
   wherein the first and second single stranded nucleic acids hybridize to the first single stranded nucleic acid linker probe of part ii) when the first single stranded nucleic acid linker probe of part ii) is not cleaved, to form a complex comprising the first single stranded nucleic acid linker probe of part ii) and the first and second single stranded nucleic acids;
f. contacting the sample with a third single stranded nucleic acid that hybridizes to a first nucleic acid sequence of the second single stranded nucleic acid linker probe of part iv) and a fourth single stranded nucleic acid that hybridizes to a second nucleic acid sequence of the second single stranded nucleic acid linker probe of part iv);
   wherein the third and fourth single stranded nucleic acids hybridize to the second single stranded nucleic acid linker probe of part iv) when the second single stranded nucleic acid linker probe of part iv) is not cleaved, to form a complex comprising the second single stranded nucleic acid linker probe of part iv) and the third and fourth single stranded nucleic acids;
g. detecting the presence or absence of the complex comprising the first single stranded nucleic acid linker probe of part ii) hybridized to the first and second single stranded nucleic acids, thereby detecting the presence or absence of nucleic acid from Chlamydia trachomatis in the sample; and
h. detecting the presence or absence of the complex comprising the second single stranded nucleic acid linker probe of part iv) hybridized to the third and fourth single stranded nucleic acids, thereby detecting the presence or absence of nucleic acid from Neisseria gonorrhoeae in the sample.

In another embodiment, the invention provides a method of detecting the presence or absence of nucleic acid from Chlamydia trachomatis in a sample from a subject, comprising
a. providing the sample from the subject;
b. contacting the sample with i) a complex comprising a CRISPR nuclease having collateral nuclease activity and a crRNA comprising a nucleic acid sequence that is complementary to a nucleic acid sequence of Chlamydia trachomatis; and ii) a single stranded nucleic acid linker probe;
c. incubating the sample with the complex of part i) and single stranded linker probe of part ii) for a period of time;
   wherein when the nucleic acid sequence of Chlamydia trachomatis is present in the sample, the collateral nuclease activity of the complex of part i) is activated and the single stranded nucleic acid linker probe or part ii) is cleaved by the nuclease;
   wherein when the nucleic acid sequence of Chlamydia trachomatis is not present in the sample, the collateral nuclease activity of the complex of part i) is not activated and the single stranded nucleic acid linker probe of part ii) is not cleaved;
d. contacting the sample with a first single stranded nucleic acid that hybridizes to a first nucleic acid sequence of the single stranded nucleic acid linker probe of part ii) and a second single stranded nucleic acid that hybridizes to a second nucleic acid sequence of the single stranded nucleic acid linker probe of part ii);
   wherein the first and second single stranded nucleic acids hybridize to the single stranded nucleic acid linker probe of part ii) when the single stranded nucleic acid linker probe of part ii) is not cleaved, to form a complex comprising the single stranded nucleic acid linker probe of part ii) and the first and second single stranded nucleic acids;
e. detecting the presence or absence of the complex comprising the single stranded nucleic acid linker probe of part ii) hybridized to the first and second single stranded nucleic acids, thereby detecting the presence or absence of nucleic acid from Chlamydia trachomatis in the sample.

In another embodiment, the invention provides a method of detecting the presence or absence of nucleic acid from Neisseria gonorrhoeae in a sample from a subject, comprising
a. providing the sample from the subject;
b. contacting the sample with i) a complex comprising a CRISPR nuclease having collateral nuclease activity and a crRNA comprising a nucleic acid sequence that is complementary to a nucleic acid sequence of Neisseria gonorrhoeae; and ii) a single stranded nucleic acid linker probe;
c. incubating the sample with the complexes of part i) and single stranded linker probe of part ii) for a period of time;
wherein when the nucleic acid sequence of Neisseria gonorrhoeae is present in the sample, the collateral nuclease activity of the complex of part i) is activated and the single stranded nucleic acid linker probe of part ii) is cleaved by the nuclease;
wherein when the nucleic acid sequence of Neisseria gonorrhoeae is not present in the sample, the collateral nuclease activity of the complex of part i) is not activated and the single stranded nucleic acid linker probe of part ii) is not cleaved;
d. contacting the sample with a first single stranded nucleic acid that hybridizes to a first nucleic acid sequence of the single stranded nucleic acid linker probe of part ii) and a second single stranded nucleic acid that hybridizes to a second nucleic acid sequence of the single stranded nucleic acid linker probe of part ii);
wherein the first and second single stranded nucleic acids hybridize to the single stranded nucleic acid linker probe of part ii) when the single stranded nucleic acid linker probe of part ii) is not cleaved, to form a complex comprising the single stranded nucleic acid linker probe of part ii) and the first and second single stranded nucleic acids;
e. detecting the presence or absence of the complex comprising the stranded nucleic acid linker probe of part ii) hybridized to the first and second single stranded nucleic acids, thereby detecting the presence or absence of nucleic acid from Neisseria gonorrhoeae in the sample.

The reaction conditions are not necessarily limiting. In some embodiments, the sample is combined at room temperature with nuclease free water (e.g., 20 uL), reaction buffer r2.1 NEB (e.g., 2 uL), 300 nM crRNA (e.g., 3 uL (30 nM)), 1 uM Cas12a (e.g., 1 uL (30 nM)), and incubated at 25 C for about 5-10 min. In some embodiments, following incubation, the single stranded target DNA and single stranded linker probe is added, mixed thoroughly, and incubated at 37 C for 5-10 min. In some embodiments, about 1 uL of proteinase K is added and mixed thoroughly, and incubated for about 10 minutes at room temperature.

In some embodiments, the methods further comprise administering to the subject an effective amount of one or more agents to treat an infection caused by Chlamydia trachomatis and/or Neisseria gonorrhoeae.

In some embodiments, when nucleic acid from Chlamydia trachomatis is present in the subject's sample, the one or more agents is selected from the group consisting of doxycycline, azithromycin, erythromycin, levofloxacin, and amoxicillin.

In some embodiments, when nucleic acid from Neisseria gonorrhoeae is present in the subject's sample, the one or more agents is selected from the group consisting of ceftriaxone, cefotaxime, ceftizoxime, ceftriaxone, gentamicine and cefixime.

The sample from the subject is not particularly limiting. In some embodiments, the subject is a male, and a urine sample is obtained from the subject. In some embodiments, the subject is a female and the sample comprises a cervical or vaginal sample, such as a secretion or swab. The biological sample obtained from the subject can be placed into a suitable solution, e.g., a buffered solution.

In some embodiments, the sample from the subject comprises a single sample, wherein detecting the presence or absence of nucleic acid from Chlamydia trachomatis and Neisseria gonorrhoeae is performed on a single sample.

In some embodiments, the sample comprises a single sample for detecting the presence or absence of both Chlamydia trachomatis and Neisseria gonorrhoeae nucleic acid. In some embodiments, the sample comprises a first sample for detecting the presence or absence of nucleic acid from Chlamydia trachomatis and a second sample for detecting the presence or absence of nucleic acid from Neisseria gonorrhoeae.

In some embodiments, the sample is treated with an agent to disrupt or lyse cells and release any nucleic acids from cells in the sample. In some embodiments, the sample can be treated by other means to disrupt cells, e.g., vortexing and homogenization. In some embodiments, the sample comprises nucleic acids that have been isolated or eluted from the sample. Such agents, techniques to disrupt cells and isolate or purify nucleic acids are well known in the art.

### CRISPR Nuclease

The CRISPR nuclease that can be used in the methods herein is not limiting. As shown herein, when the nuclease is associated with a CRISPR RNA (crRNA), they can specifically complement with a target sequence from Chlamydia trachomatis or Neisseria gonorrhoeae, inducing enzymatic cleavage of both the targeted sequence and nontargeted collateral cleavage of all nucleic acids (e.g., ssDNA) in the environment. In some embodiments, the CRISPR nuclease to be used has collateral nuclease activity.

CRISPR-Cas systems include Types I, II, III, IV, V, and VI systems. In some embodiments, the CRISPR-Cas system can be a Type V CRISPR-Cas system (e.g., Cas12a, previously termed Cpf1). The CRISPR-Cas12a nuclease used in the methods described herein can have at least about 70%, at least about 75%, at least about 80%, at least about 85%, at least about 90%, at least about 95%, at least about 97%, at least about 99% sequence identity to a wild-type CRISPR-Cas12a nuclease.

Cas molecules of a variety of species can be used in the methods and compositions described herein. In some embodiments, the Cas nuclease is from Staphylococcus aureus. In some embodiments, the Cas nuclease is from S. pyogenes, S. thermophiles, or Neisseria meningitides. Additional Cas species include: Acidovorax avenae, Actinobacillus pleuropneumoniae. Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis. Streptococcus sp., Subdoligranulum sp., Tistrella mobilis, Treponema sp., or Verminephmrobacter eiseniae.

A Cas molecule, as that term is used herein, refers to a molecule that can interact with a crRNA molecule and, in concert with the crRNA molecule, localize (e.g., target or home) to a site which comprises a target domain and PAM sequence.

The Cas molecule is capable of cleaving a target nucleic acid molecule. The ability of a Cas molecule to interact with and cleave a target nucleic acid is PAM sequence dependent. A PAM sequence is a sequence in the target nucleic acid. In an embodiment, cleavage of the target nucleic acid occurs upstream from the PAM sequence. Cas molecules from different bacterial species can recognize different sequence motifs (e.g., PAM sequences). In some embodiments, the PAM motif comprises TTTC. In some embodiments, The PAM sequence requirement for Cas12a is “TTN/TTTN/TTTV.” The ability of a Cas molecule to recognize a PAM sequence can be determined, e.g., using a transformation assay described in Jinek et al., Science 2012, 337:816.

In one embodiment, the Cas12a molecule is from an organism selected from Lachnospiraceae bacterium ND2006, Moraxella bovoculi Francisella tularensis subsp. novicida U112, Acidaminococcus sp. BV3L6, Planctomycetota bacterium, Deltaproteobacteria bacterium, Lachnospiraceae bacterium MA2020 and combinations thereof. In some embodiments, Lachnospiraceae bacterium ND2006 Cas12a has the amino acid sequence of SEQ ID NO:35 (Accession No. OK557998.1. In some embodiments, the nucleotide sequence of Cas12a is SEQ ID NO:36 and is codon optimized. In some embodiments, the Cas12a is available commercially (e.g., from New England BioLabs, Inc., see Cat. Nos. M0653S and M0653T).

In some embodiments, the PAM sequence requirement for Cas12a is “TTN/TTTN/TTTV”. (N=A/T/C/G; V=A/C/G). In some embodiments, the Cas12a is FnCas12a (from Francisella novicida), LbCas12a (from Lachnospiraceae bacterium), or AsCas12a (from Acidaminococcus sp.).

In some embodiments, the nucleic acid sequence of Cas12a contains a nucleotide sequence that is highly identical, at least 90% identical, with a nucleotide sequence encoding Cas12a polypeptide. In some embodiments, the nucleic acid sequence of Cas12a comprises a nucleotide sequence that is at least 60%, 65%, 70%, 75%, 80%, 85%, 90%, 91%, 92%, 93%, 94%, 95%, 96%, 97%, 98% 99% or 100% identical with the encoding nucleotide sequence set forth in SEQ ID NO:36.

When a Cas12a polynucleotide is used for the production of Cas12a polypeptide, the polynucleotide may include the coding sequence for the full-length polypeptide or a fragment thereof, by itself; the coding sequence for the full-length polypeptide or fragment in reading frame with other coding sequences, such as those encoding a leader or secretory sequence, a pre-, or pro or prepro-protein sequence, nuclear localization signal or other fusion peptide portions. The polynucleotide may also contain non-coding 5′ and 3′ sequences, such as transcribed, non-translated sequences, splicing and polyadenylation signals, ribosome binding sites and sequences that stabilize mRNA.

In some embodiments, the nucleotide sequence encoding Cas12a or a biologically active fragment or derivative thereof includes nucleic acid molecules comprising a polynucleotide having a nucleotide sequence at least 90%, 91%, 92%, 93%, 94%, 95%, 96%, 97%, 98% 99% or 100% identical to (a) a nucleotide sequence encoding Cas12a having the amino acid sequence in SEQ ID NO:35 (b) a nucleotide sequence complementary to the nucleotide sequences in (a).

In some embodiments, the nucleotide sequences are at least 90% identical over their entire length to a polynucleotide encoding a Cas12a having the amino acid sequence set out in SEQ ID NO:35; and polynucleotides which are complementary to such polynucleotides. In some embodiments, the polynucleotides are at least 91%, 92%, 93%, 94%, 95%, 96%, 97%, 98%, or at least 99% identical. In some embodiments, the nucleic acid molecule encodes a biologically active fragment of Cas12a protein.

In some embodiments, a Cas12a molecule comprises an amino acid sequence having at least 60%, 65%, 70%, 75%, 80%, 85%, 90%, 95%, 96%, 97%, 98%, or 99% homology with SEQ ID NO:35 or a naturally occurring Cas12a molecule sequence, e.g., a Cas12a molecule from a species listed herein.

In some embodiments, the Cas12a protein comprises an amino acid sequence that differs from a sequence of SEQ ID NO:35 by as many as 1, but no more than 2, 3, 4, or 5 residues.

Naturally occurring Cas molecules possess a number of properties, including: nickase activity, nuclease activity (e.g., endonuclease and/or exonuclease activity); helicase activity; the ability to associate functionally with a crRNA molecule; and the ability to target (or localize to) a site on a nucleic acid (e.g., PAM recognition and specificity). In some embodiments, a Cas molecule can include all or a subset of these properties. In typical embodiments, Cas molecules have the ability to interact with a crRNA molecule and, in concert with the crRNA molecule, localize to a site in a nucleic acid. Other activities, e.g., PAM specificity, cleavage activity, or helicase activity can vary more widely in Cas molecules.

Cas molecules with desired properties can be made in a number of ways, e.g., by alteration of a parental, naturally occurring Cas molecule to provide an altered Cas molecule having a desired property. One or more mutations or differences relative to a parental Cas molecule can be introduced. Such mutations and differences can comprise: substitutions (e.g., conservative substitutions or substitutions of non-essential amino acids); insertions; or deletions. In some embodiments, a Cas molecule can comprises one or more mutations or differences, e.g., at least 1, 2, 3, 4, 5, 10, 15, 20, 30, 40 or 50 mutations but less than 200, 100, or 80 mutations relative to a reference Cas9 molecule.

crRNAs

A crRNA molecule, as that term is used herein, refers to a nucleic acid that promotes the specific targeting or homing of a crRNA molecule/Cas molecule complex to a target nucleic acid.

The crRNA molecule can be unimolecular (having a single RNA molecule), sometimes referred to herein as “chimeric” gRNAs, or modular (comprising more than one, and typically two, separate RNA molecules). In one embodiment, the crRNA molecule can be used with a Cas protein from Staphylococcus aureus.

The crRNA comprises a targeting domain (which is complementary to the target nucleic acid) and other sequences that are necessary to bind Cas. The targeting domain comprises a nucleotide sequence that is complementary, e.g., at least 80, 85, 90, or 95% complementary, e.g., fully complementary, to the target sequence on the target nucleic acid. The targeting domain is part of an RNA molecule and will therefore comprise the base uracil (U), while any DNA encoding the crRNA molecule will comprise the base thymine (T). While not wishing to be bound by theory, it is believed that the complementarity of the targeting domain with the target sequence contributes to specificity of the interaction of the crRNA molecule/Cas molecule complex with a target nucleic acid. It is understood that in a targeting domain and target sequence pair, the uracil bases in the targeting domain will pair with the adenine bases in the target sequence. In an embodiment, the target domain itself comprises, in the 5′ to 3′ direction, an optional secondary domain, and a core domain. In an embodiment, the core domain is fully complementary with the target sequence. In an embodiment, the targeting domain is 5 to 50, 10 to 40, e.g., 10 to 30, e.g., 15 to 30, e.g., 15 to 25 nucleotides in length. In an embodiment, the targeting domain is 15, 16, 17, 18, 19, 20, 21, 22, 23, 24 or 25 nucleotides in length. The strand of the target nucleic acid with which the targeting domain is complementary is referred to herein as the complementary strand. Some or all of the nucleotides of the domain can have a modification, e.g., a modification described herein. Guidance on the selection of targeting domains can be found, e.g., in Fu et al., Nat Biotechnol 2014 (doi: 10.1038/nbt.2808) and Sternberg S H et al., Nature 2014 (doi: 10.1038/nature13011).

In Type V CRISPR-Cas systems, a crRNA sequence forms a duplex. The duplex binds the CRISPR-Cas nuclease such that the crRNA and the CRISPR-Cas nuclease form a complex. The crRNA thus directs the activity of the CRISPR-Cas nuclease and provides specificity in targeting the CRISPR-Cas nuclease to the targeted genomic location.

Each crRNA comprises a spacer sequence that defines the target sequence of a target nucleic acid (or target locus). The “target sequence” is adjacent to a PAM sequence in the target DNA and is cleaved by the CRISPR-Cas nuclease. The “target nucleic acid” is a double-stranded molecule; one strand comprises the target sequence and is referred to as the “PAM strand,” and the other complementary strand is referred to as the “non-PAM strand.” One of skill in the art recognizes that the crRNA spacer sequence hybridizes to the reverse complement of the target sequence, which is located in the non-PAM strand of the target nucleic acid. The spacer of a crRNA interacts with a target nucleic acid in a sequence-specific manner via hybridization (i.e., base pairing). The nucleotide sequence of the spacer thus varies depending on the target sequence of the target locus.

In some embodiments, the spacer sequence of the crRNA may be from about 15 to about 30 nucleotides long, or about 18 to 22 nucleotides long. In some embodiments, the spacer sequence is 15, 16, 17, 18, 19, 20, 21, 22, 23, 24, 25, 26, 27, 28, 29, or 30 nucleotides. In some embodiments, a spacer sequence is 18, 19, 20, or 21 nucleotides long. In some embodiments, the spacer sequence is 20 nucleotide long. In some embodiments, the spacer sequence has at least about 90%, at least about 95%, or at least about 99% sequence identity to the target sequence in the target nucleic acid. In certain embodiments, the spacer sequence has 100% sequence identity to the target sequence.

In some embodiments, the crRNA is complementary to a nucleic acid sequence of a target gene. In some embodiments, the target gene of Chlamydia trachomatis is cryptic plasmid ORF6 segment sequence and the target gene of Neisseria gonorrhoeae is a major outer membrane protein porB.

In some embodiments, the nucleic acid of Chlamydia trachomatis that the crRNA is complementary to is to a fragment of a cryptic plasmid ORF6 segment sequence. In some embodiments, the cryptic plasmid ORF6 segment sequence has an amino acid sequence comprising SEQ ID NO:1 and a nucleotide sequence comprising SEQ ID NO:2.

In some embodiments, the target sequence in Chlamydia trachomatis comprises any of SEQ ID NOS:6-9. In some embodiments, the crRNA that is complementary to the fragment of cryptic plasmid ORF6 segment sequence comprises SEQ ID NO:10.

In some embodiments, the nucleic acid of Neisseria gonorrhoeae that the crRNA is complementary to is to a fragment of a major outer membrane protein porB sequence. In some embodiments, the porB sequence has an amino acid sequence comprising SEQ ID NO:13 and a nucleotide sequence comprising SEQ ID NO:14.

In some embodiments, the target sequence in Neisseria gonorrhoeae comprises any of SEQ ID NOS:18-23. In some embodiments, the crRNA that is complementary to the fragment of porB sequence comprises SEQ ID NO:24.

### Single Stranded Linker Probes

The single stranded nucleic acid linker probes for use in detecting the presence or absence of Chlamydia trachomatis and Neisseria gonorrhoeae nucleic acid in the sample are not limiting.

In some embodiments, the single stranded linker probe does not comprise a sequence of a target gene or nucleic acid of Chlamydia trachomatis or Neisseria gonorrhoeae and is unrelated to nucleic acids of these organisms.

In some embodiments, for detection of the presence or absence of nucleic acid from Chlamydia trachomatis, the single stranded linker probe comprises a nucleic acid sequence of a target gene or nucleic acid of Chlamydia trachomatis. In some embodiments, for detection of the presence or absence of nucleic acid from Neisseria gonorrhoeae, the single stranded linker probe comprises a nucleic acid sequence of the target gene or nucleic acid of Neisseria gonorrhoeae.

In some embodiments, the nucleic acid sequence of the single stranded linker probe does not comprise a nucleic acid sequence that is complementary to the crRNA.

In some embodiments, the nucleic acid sequence of the single stranded linker probe comprises a sequence of the target gene, but does not overlap with the sequence that is complementary to the crRNA.

In some embodiments, the single stranded nucleic acid linker probes comprise a nucleic acid sequence that is complementary to a nucleic acid sequence of the Chlamydia trachomatis or Neisseria gonorrhoeae crRNA.

In some embodiments, the probe ranges from about 40-120 nucleotides in length. In some embodiments, the probe is about 40, 45, 50, 55, 60, 65, 70, 75, 80, 85, 90, 95, 100, 105, 110, 115, or about 120 nucleotides in length. In some embodiments, the probes are single stranded DNA.

In some embodiments, the single stranded nucleic acid linker probe for use in detecting the presence or absence of Chlamydia trachomatis nucleic acid comprises SEQ ID NO:5.

In some embodiments, the single stranded nucleic acid linker probe for use in detecting the presence or absence of Neisseria gonorrhoeae nucleic acid comprises SEQ ID NO:17.

In some embodiments, the single stranded nucleic acid linker probe is DNA.

### Single Stranded Nucleic Acids

In accordance with some embodiments, the sample comprising the complex comprising a CRISPR nuclease, crRNA and single stranded nucleic acid linker probe is contacted with single stranded nucleic acids that are capable of hybridizing to a single stranded nucleic acid linker probe that has not been cleaved. In some embodiments, one of the single stranded nucleic acids hybridizes to a 5′ region of the single stranded nucleic acid linker probe and another single stranded nucleic acid hybridizes to a 3′ region of the single stranded nucleic acid linker probe.

The length of the single stranded nucleic acids is not particularly limiting and in some embodiments are 15-40 nucleotides in length. In some embodiments, the single stranded nucleic acids are about 20 nucleotides in length. In some embodiments, the single stranded nucleic acids are DNA.

In some embodiments, the binding energy of the single stranded nucleic acids is more negative than about −8.0 kcal/mol. In some embodiments, the binding energy of the single stranded nucleic acids is more negative than about −9.0 kcal/mol. In some embodiments, the binding energy of the single stranded nucleic acids is more negative than about −10.0 kcal/mol. In some embodiments, the binding energy of the single stranded nucleic acids is more negative than about −11.0 kcal/mol.

In some embodiments, at least a pair of single stranded nucleic acids for use in detecting the presence or absence of Chlamydia trachomatis nucleic acid comprises SEQ ID NOS:3 and 4.

In some embodiments, at least a pair of single stranded nucleic acids for use in detecting the presence or absence of Chlamydia trachomatis nucleic acid comprises SEQ ID NOS:31 and 32.

In some embodiments, at least a pair of single stranded nucleic acids for use in detecting the presence or absence of Neisseria gonorrhoeae nucleic acid comprises SEQ ID NOS:15 and 16.

In some embodiments, at least a pair of single stranded nucleic acids for use in detecting the presence or absence of Neisseria gonorrhoeae nucleic acid comprises SEQ ID NOS:33 and 34.

In some embodiments, one or more of the single stranded nucleic acids are labeled. The label is not limiting. In some embodiments, the label is selected from any one of a fluorescent label, radiolabel, streptavidin, an antibody, 6-carboxyfluorescein (FAM), biotin, a thiol group located on the 5′ or 3′ end of the single stranded nucleic acid, a nanoparticle, and combinations thereof. In some embodiments, the nanoparticle comprises gold. In some embodiments, the nanoparticle is a plasmonic nanoparticle. In some embodiments the fluorescent label is FITC.

In some embodiments, one single stranded nucleic acid of the pair is labeled. In some embodiments, both are labeled.

In some embodiments, one single stranded nucleic acid of the pair is labeled on a 5′ end of the molecule, while the other single stranded nucleic acid of the pair is labeled on the 3′ end of the molecule. In some embodiments, one single stranded nucleic acid of the pair is conjugated with a thiol group and/or 6-carboxyfluorescein on the 5′ end of the nucleic acid and the other single stranded nucleic acid of the pair is conjugated with a thiol group and/or 6-carboxyfluorescein on the 3′ end of the molecule.

In some embodiments, one single stranded nucleic acid of the pair is conjugated with a thiol group and/or biotin on the 5′ end of the nucleic acid and the other single stranded nucleic acid of the pair is conjugated with a thiol group and/or 6-carboxyfluorescein on the 3′ end of the molecule.

In some embodiments, one single stranded nucleic acid of the pair is conjugated with a thiol group and/or 6-carboxyfluorescein on the 5′ end of the nucleic acid and the other single stranded nucleic acid of the pair is conjugated with a thiol group and/or biotin on the 3′ end of the molecule.

In some embodiments, one single stranded nucleic acid of a pair is bound to a gold nanoparticle and the second single stranded nucleic acid of the pair is bound to a gold nanoparticle.

In some embodiments, a single gold nanoparticle can be bound to more than one single stranded nucleic acids of the same sequence, or of different sequences.

In some embodiments, the hybridization of the single stranded nucleic acid linker probe to the single stranded nucleic acids form agglomerates. In some embodiments, the sample comprising the agglomerates has a different absorbance measurement than a sample that lacks agglomerates. In some embodiments the absorbance measurement is at 520 nm. In some embodiments, the absorbance is done using UV-Vis spectroscopy.

In some embodiments, one single stranded nucleic acid is bound to 6-carboxyfluorescein (FAM) and the other single stranded nucleic acid of the pair is bound to biotin.

### Devices/Assays and Kits

In some embodiments, methods can be used with various devices such as lateral flow devices. In some embodiments, the methods comprise loading the sample comprising the single stranded linker probes and the single stranded nucleic acids onto a lateral flow assay to detect the presence or absence of the complex comprising the single stranded nucleic acid linker probes hybridized to the single stranded nucleic acids. In some embodiments, the lateral flow assay is a dipstick.

In some embodiments, the lateral flow assay comprises streptavidin in a test strip. In some embodiments an antibody is added to the sample that binds one or more labels on the single stranded nucleic acid. In some embodiments, the antibody is conjugated to a detectable label. In some embodiments, the detectable label is selected from a fluorophore, radiolabel, and nanoparticle. In some embodiments, the fluorophore is FITC.

In some embodiments, the nanoparticle is a gold nanoparticle.

In some embodiments utilizing a lateral flow immunoassay format, the lateral flow device may comprise two ports: a sample port, which is positioned between a conjugate pad (containing a labeled analyte-binding partner) and a test site or line (containing an immobilized analyte-binding partner) and a chase port, which is positioned downstream (e.g. toward the end of the device away from test site) of the conjugate pad. In such devices comprising two ports, sample is deposited downstream of the test site via the sample port and fluid flow through the conjugate pad is initiated by depositing solution (e.g. diluent, buffer, or the like) via the chase port. The “chase” solution dissolves the labeled reagents in the conjugate pad and flows through to interact with the sample and then the immobilized reagents at the test site. Alternatively, sample is deposited downstream of the test site via the sample port and migrates toward the test site(s), resulting in the capture of the analyte(s) by the immobilized agents at the test site(s). Subsequently, fluid flow through the conjugate pad is initiated by depositing solution (e.g. diluent, buffer, or the like) via the chase port. The “chase” solution dissolves the labeled reagents in the conjugate pad and flows through to interact with the captured analyte(s) at the test site(s).

In other embodiments utilizing the lateral flow immunoassay format, the lateral flow device may comprise one port: a sample/chase buffer port, which is positioned below a conjugate pad (containing a labeled analyte-binding partner) and the test site(s) or line(s) containing immobilized analyte-binding partner(s). In such devices, the sample is deposited downstream of the test site via the sample/buffer port and fluid flow through the conjugate pad is initiated by depositing solution (e.g. diluent, buffer, or the like) via the sample/chase buffer port. The “chase” solution dissolves the labeled reagents in the conjugate pad and flows through to interact with the sample and then the immobilized reagents at the test site(s).

In another embodiment, the invention provides a kit, comprising
a. one or more test strips, one or more solid supports, one or more beads, one or more flow paths in a lateral flow immunoassay device, and/or one or more wells in a microtiter plate;
b. a CRISPR nuclease having collateral nuclease activity;
c. a crRNA comprising a nucleic acid sequence that is complementary to a nucleic acid sequence of Chlamydia trachomatis, a first single stranded nucleic acid linker probe, a first single stranded nucleic acid that is complementary to a first nucleic acid sequence of the first single stranded nucleic acid linker probe and a second single stranded nucleic acid that is complementary to a second nucleic acid sequence of the first single stranded nucleic acid linker probe;
d. a crRNA comprising a nucleic acid sequence that is complementary to a nucleic acid sequence of Neisseria gonorrhoeae, a second single stranded nucleic acid linker probe, a first single stranded nucleic acid that is complementary to a first nucleic acid sequence of the second single stranded nucleic acid linker probe and a second single stranded nucleic acid that is complementary to a second nucleic acid sequence of the second single stranded nucleic acid linker probe;
e. optionally one or more solutions suitable for use; and
f. optionally instructions for use.

In another embodiment, the invention provides a kit, comprising
a. one or more test strips, one or more solid supports, one or more beads, one or more flow paths in a lateral flow immunoassay device, and/or one or more wells in a microtiter plate;
b. a CRISPR nuclease having collateral nuclease activity;
c. a crRNA comprising a nucleic acid sequence that is complementary to a nucleic acid sequence of Chlamydia trachomatis, a single stranded nucleic acid linker probe, a first single stranded nucleic acid that is complementary to a first nucleic acid sequence of the single stranded nucleic acid linker probe and a second single stranded nucleic acid that is complementary to a second nucleic acid sequence of the single stranded nucleic acid linker probe;
d. optionally one or more solutions suitable for use; and
e. optionally instructions for use.

In another embodiment, the invention provides a kit, comprising
a. one or more test strips, one or more solid supports, one or more beads, one or more flow paths in a lateral flow immunoassay device, and/or one or more wells in a microtiter plate;
b. a CRISPR nuclease having collateral nuclease activity;
c. a crRNA comprising a nucleic acid sequence that is complementary to a nucleic acid sequence of Neisseria gonorrhoeae, a single stranded nucleic acid linker probe, a first single stranded nucleic acid that is complementary to a first nucleic acid sequence of the single stranded nucleic acid linker probe and a second single stranded nucleic acid that is complementary to a second nucleic acid sequence of the single stranded nucleic acid linker probe;
d. optionally one or more solutions suitable for use; and
e. optionally instructions for use.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. Development of a Multiplex Crispr Based Assay

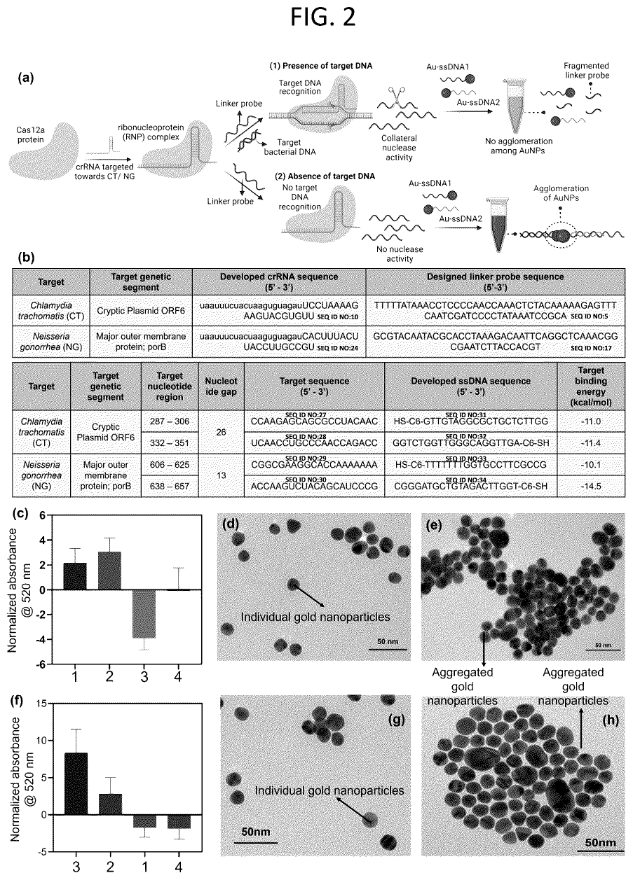

Based on our previous experience of designing multiple targeted ssDNAs for selective sensing of COVID-19 causative virus, SARS-CoV-2 and various other bio-analytes in living cells, we have already identified the unique crRNAs targeted towards specific genetic segments of CT and NG respectively (Moitra et al., ACS Nano, (2020), 14:7617-7627; Alafeef et al., ACS Nano, (2020), 14:17028-17045; Alafeef et al., Nature Protocols, (2021), 16:3141-3162; Dighe et al., Biosensors and Bioelectronics, (2022), 200:13900; Alafeef et al., ACS Nano, (2021), 15:13742-13758; Moitra et al., Chemical Communications, (2021), 57:6229-6232; Sheffield et al. Nanoscale, (2022), 14:5112-5120; Moitra et al., Biosensors and Bioelectronics, (2022), 208:114200). While we target cryptic plasmid ORF6 segment for CT, we choose major outer membrane protein, porB, for NG for the design and development of crRNA. (FIG. 2a) This crRNA sequence is perfectly suited for activation of CRISPR/Cpf1 or Cas12a so that upon detection of the target DNA sequence, its collateral nuclease activity gets stimulated. We have further designed two of the ssDNAs targeted towards the same genetic sequences of CT and NG. The corresponding target binding and binding site disruption energies are highlighted in FIG. 2b. A complimentary single-stranded DNA sequence noted as linker probe, is also developed which covers both ssDNA regions. The unique sequences for crRNA and ssDNAs are mentioned in FIG. 2b. These ssDNAs are then differentially functionalized in two ways: (i) to characterize the agglomeration phenomena among linker probe and ssDNAs, we have first modified ssDNA1 at 5′-end and ssDNA2 at 3′-end with thiol moieties; (ii) further to include the ssDNAs directly in dipstick based lateral flow assay, we have modified ssDNA1 at 5′-end by biotin and ssDNA2 at 3′-end with 6′-FAM. In presence of the target DNA, the ribonucleoprotein (RNP) complex turns its collateral cleavage activity ‘ON’ and cleaves the suitably designed linker probe present in the suspension. Hence, it is expected that in presence of the target CT/NG DNA in the suspension, the linker probe gets chopped off, while in absence of target DNA, the linker probe remains intact. Labeled ssDNA will bind with their complementary probe sequence depending on whether target DNA is present. Before transitioning to the lateral flow-based experiment, we first proved the phenomena through the agglomeration among the ssDNAs and linker probe while modifying the ssDNAs by thiol moieties either at 5′- or 3′-ends and then conjugating them with gold nanoparticles. The differential aggregation among the gold nanoparticles represents the presence of target DNA (FIG. 2a). In this study, we first utilized DNA extracted from deidentified cervical swab and urine samples as the input target sequence. We analyzed 60 samples (including both urine and cervical swab samples) out of which 15 were CT+, 15 were both NG+ and CT+, 15 were NG+ and 15 were both negative (as validated using gold standard PCR). In presence of the target bacterial DNA, thiolated ssDNA-capped AuNPs do not agglomerate and thus demonstrate no change in its surface plasmon resonance (SPR). However, the agglomeration of ssDNA-capped AuNPs in the absence of bacterial DNA was studied by UV-visible absorbance spectroscopy (UV-Vis) and transmission electron microscopic (TEM) studies (FIG. 2c-h). Thus, the current study utilizes the intrinsic optical properties of plasmonic AuNPs to demonstrate the targeting ability of the crRNA and ssDNAs for CT & NG. The specificity and selectivity of the developed platform has also been determined. Moreover, the study established the targeting capabilities of the new DNA probes and gave us confidence in the development of lateral flow assays.

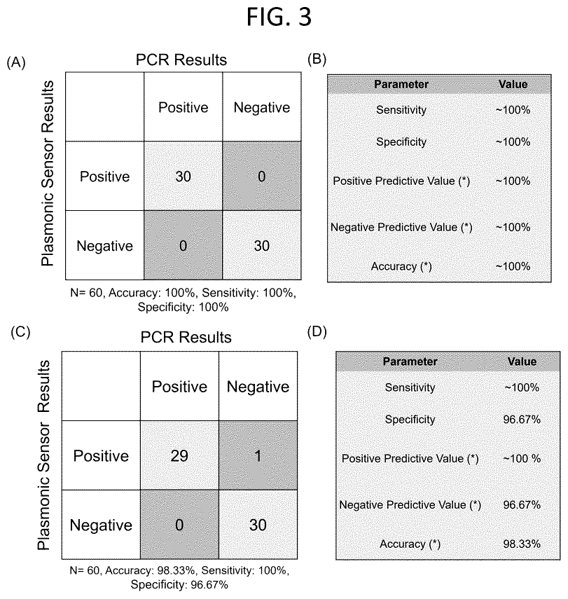

Generally, the nucleic acid-based detection of bacterial species involves the isolation and purification of DNA which consists of multiple steps. POC assays are ideal when the DNA isolation can be accomplished easily, rapidly, and preferably in one step. As a result, we used the enzymatic DNA/RNA extraction buffer from CHAI directly to the source media to detect CT and NG from the infected media without purification. For preliminary studies, we analyzed 40 de-identified samples (including both urine and cervical swab samples, N=10 for CT+, NG+, CT & NG both +, CT & NG both − (as validated using gold standard PCR) (FIG. 3). Validating the results against gold standard PCR revealed good agreement. Further, cervical swabs performed better than urine specimens in terms of sensitivity.

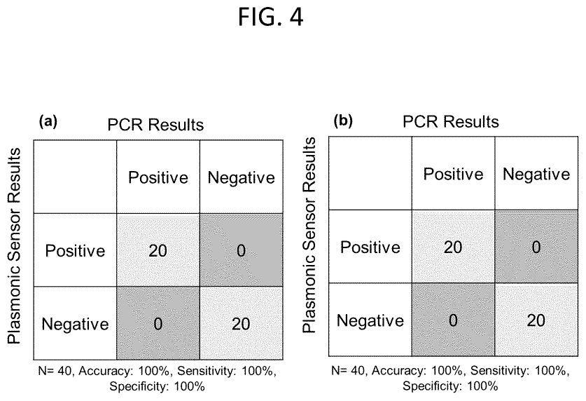

It is fundamentally necessary to develop a point-of-care diagnostic test with reasonable specificity and sensitivity, which can be implemented immediately without the need for any supporting equipment. Thus, in order to make this an effective point-of-care assay, we are using readily available universal dipstick strips (Milenia Biotec—Hybrid Detect). In presence of the target DNA, Cas-dependent reporter cleavage leads to the fragmentation of the linker probe. The ssDNA probes cannot bind to the fragmented linker probe and cannot show any test band. However, in absence of the target DNA, the labeled ssDNAs bind with the linker probe and show a prominent test band. Thus, the developed platform can differentiate between intact and cleaved reporter/linker probes and hence can selectively detect the presence of CT/NG DNA in a suspension (FIG. 4).

1. Develop, optimize, and analytically validate one-step CRISPR-Cas12 based assay for the multiplexed diagnosis of CT and NG. This part will optimize the targeted crRNAs, linker probe and appropriately labeled ssDNAs. We will validate our assay with frozen CT and NG strains isolated from cervical swabs and urine samples. The sensitivity, specificity, time of response and limit of detection of the assay will also be determined. The cross-interference of the assay will be determined against other bacterial pathogens. We will then evaluate the assay performance with the direct addition of one-step DNA extraction buffer and without any separate DNA isolation and purification steps. The results will further be optimized in terms of concentration of crRNA, linker probe, and ssDNAs.

1a: Optimal probe design for CT and NG.

Rationale: We have already developed CT and NG targeted crRNA, linker probe and ssDNAs. The ssDNAs are also suitably labeled depending on the need, i.e., thiolated at their 5′-end to prove the agglomeration phenomena in presence/absence of their target DNA. In this part, we will evaluate the binding efficiency and the agglomeration of ssDNAs with linker probe by optimizing the concentration of the DNA probes.

To characterize the aggregation kinetics among the ssDNAs and linker probe, the thiolated ssDNAs will be conjugated with citrate stabilized AuNPs. To improve the agglomeration among the gold nanoparticles, we will employ a previously developed technique in our laboratory (Moitra et al., ACS Nano, (2020), 14:7617-7627; Alafeef et al., ACS Nano, (2020), 14:17028-17045; Alafeef et al. Nature Protocols, (2021), 16:3141-3162; Dighe el al, Biosensors and Bioelectronics, (2022), 200:113900; Alafeef et al, ACS Nano, (2021), 15:13742-13758; Moitra et al, Chemical Communications, (2021), 57:6229-6232; Sheffield et al, Nanoscale, (2022), 14:5112-120; Moitra et al. Biosensors and Bioelectronics, (2022). 208:114200). Accordingly, we will differentially modify the ssDNAs, one at 5′-end and the other at 3′-end with thiol moieties. As the nuclease activity of RNP complex is active in the presence of target DNA, it is presumed that the linker probe will fragment in the presence of the target DNA (Wang et al, Analytica Chimica Acta, (2021), 1185: 338882; Nouri et al, Biosensors and Bioelectronics, (2021), 178:113012; Ma et al, ACS Sensors 2021, 6(8), 2920-2927; He et al, Biosensors and Bioelectronics, (2022). 198:113857). Due to the reduced aggregation potential, the AuNPs will not show any significant modification in their plasmonic response. In the absence of target DNA, however, RNP complex does not engage in collateral cleavage, keeping the linker probe intact. It will facilitate the agglomeration of gold nanoparticles and demonstrate a noticeable change in the suspension's absorbance and color (FIG. 2a).

As part of our studies, we have already identified the crRNA sequences to selectively target CT and NG. The designed linker probe and suitably modeled ssDNA sequences are also highlighted in FIG. 2b. Currently, the crRNA for CT targets the cryptic plasmid ORF6 segment and crRNA for NG targets major outer membrane protein, porB. The reason behind the choice of ORF6 for CT is ORF6/pgp4 (transcriptional regulator of virulence associated genes) presented the highest mean expression among all CT strains, followed by the virulence factor ORF5/pgp3 (also regulated by ORF6/pgp4) (Ferreira et al., Microbiological Research, (2013), 168:333-339). Further, the analytical sensitivity of cryptic plasmid was found to be better than other genetic segments for CT(Realtime CT/NG Assay|Abbott Molecular www.molecular.abbott/us/en/products/infectious-disease/realtime-ct-ng (accessed 2022 Oct. 3). On the other hand, Neisseria gonorrhoeae has two porins, PIA and PIB, whose genes (porA and porB, respectively) are alleles of a single por locus (Geraats-Peters et al., Journal of Clinical Microbiology, (2005). 43:5653-5659; Olesky et al, Journal of bacteriology, (2006), 188: 2300-2308. We herein considered porB as our target gene for NG as mutations in PIB have no effect on antibiotic permeation alone (Ng et al., Canadian Journal of Infectious Diseases and Medical Microbiology, (2005), 16:15-25).

Now, for feasibility, the proposed aggregation-induced sensing principle has been demonstrated with bacterial DNAs isolated and purified from CT and NG clinical samples (obtained from Bocabiolistics and Carle Illinois medical facility). We observed the UV-Visible absorbance of AuNPs at 520 nm. It is presumed that the absorbance of AuNPs will remain intact in case of positive target DNA samples, however, it will be compromised or shifted largely from 520 nm in absence of the target CT/NG DNA. To our expectations, when the suspension having crRNA targeted for CT/NG, linked probe and ssDNA conjugated AuNPs were added with respective DNAs from CT +ve, CT & NG both +ve, NG +ve and both −ve samples, the results matched (FIG. 2c, f). It has been hypothesized that in presence of target CT/NG DNA, the AuNPs will be separated from each other while in absence of target CT/NG DNA, they will be agglomerated to bathochromically shift the absorbance from 520 nm. This hypothesis has then been corroborated with the observation obtained from transmission electron microscopy. In presence of target DNA, distributed pattern of AuNPs was observed (FIG. 2d, g), while in absence of target CT/NG DNA, the AuNPs were found to be largely aggregated (FIG. 2e, h). The results were also corroborated with enhanced dark-field hyperspectral imaging microscope. The sensing probes, decorated for CT, are found to be highly specific for CT DNA from the hyperspectral analyses. However, under this part, we will validate this observation from multiple CT/NG positive/negative DNA samples. In order to maximize the agglomeration among gold nanoparticles, the concentration of the reagent probes will be optimized because it is predicted that i) the concentration of Cas12 protein will increase, ii) the concentration of crRNA will decrease, or iii) the concentration of linker probe will increase, and iv) the gold nanoparticle concentration will reach its optimal concentration.

The assay will be validated against different CT and NG DNA samples isolated from cervical swabs and urine specimens with a standardized concentration of linker probe and ssDNA conjugated gold nanoparticles.

The assay activity is largely dependent on the selectivity of crRNA in the detection of the target CT and NG DNA. A successful assay will rely heavily on the concentrations of Cas12, crRNA, linker probes, as well as AuNPs conjugated with thiolated ssDNAs.

1b: Analytically evaluate the assay sensitivity and specificity.

The sensitivity, specificity, and limit of detection (LOD) of the assay needs to be analytically evaluated to justify the assay performance towards minute concentration of CT and NG gene.

The concentrations of CT and NG DNA will be titrated to observe the effect on plasmon response in order to evaluate the analytical sensitivity of the assay. Towards this, we will use the genomic DNA of CT and NG available from ATCC. Our data showed that the assay remained active at 0.08 ng/L of target NG genomic DNA. However, under this part, it will be optimized for CT as well. On the other hand, the DNA isolated from fresh and frozen clinical samples of CT and NG will also be considered. Their concentrations will be normalized to copies/mL in suspension buffer and studied under this aim to evaluate the sensitivity and LOD of the assay. As a proof-of-concept study, we have analyzed 60 clinical samples where 25 of them are CT +ve, 25 are both CT and NG +ve, 25 are NG +ve and 25 are both CT and NG −ve. The results as obtained from CT and NG assay in the form of confusion matrix are highlighted in FIGS. 3a and 3c respectively. The sensitivity, specificity, accuracy, positive predictive value and negative predictive values were then calculated from the obtained data and represented in FIGS. 3b and 3d respectively. However, under this part, we will undertake a large pool of clinical samples and intend to establish the aforementioned sensing parameters to a value >98%.

Further, the cross-interference of the assay will be determined with a specific concentration of DNA obtained from fresh cultures of Staphylococcus aureus, Acinetobacter baumannii, Escherichia coli, Bacillus subtilis, Streptococcus mutans bacteria. As a part of our preliminary study, we have also conducted this experiment. This cross-reactivity study with a bacteria panel (as mentioned above) demonstrated that the crRNA is highly selective only towards its target CT and NG DNA. However, under this part, we will encounter other interfering bacterial and viral species (e.g., Treponema pallidum—Syphilis, Human papillomavirus (HPV), Herpes simplex virus (HSV)) and evaluate the assay performance in the assay media.

The sensitivity, specificity, interference and LOD of the assay will be determined. Specificity of the novel probes will be established.

The developed crRNA is anticipated to be highly specific towards CT and NG strain. The sensitivity of the assay will be dependent on the suitable adjustment of the concentration of different assay reagents including crRNA, linker probe and ssDNAs.

1c: Standardize the assay performance with a one-Step DNA extraction buffer.

Generally, the nucleic acid-based detection of bacterial species involves the isolation and purification of DNA which consists of multiple steps. An ideal POC assay would be the one where the DNA isolation can be achieved in an easy and rapid manner, preferably in one step.

In this part, we will standardize our assay performance without isolating and purifying the bacterial DNA. Our hypothesis was that the assay depends solely on the presence of target DNA and does not affect interfering elements. The enzymatic DNA/RNA extraction buffer, readily available from CHAI, will therefore be added directly to the source media (Lever et al., Frontiers in Microbiology, (2015), 6; Stinson et al., Letters in Applied Microbiology, (2018), 68:2-8). The hypothesis is that no steps of heating, centrifuging, or mechanical agitation are required. The cells should be exposed to lysis buffer at ambient temperature for 10 minutes. In the case of difficult-to-lyse cells, vortexing or homogenization can improve extraction efficiency. Solid samples, like cervical swabs, will be buffered directly with the 1× buffer, while liquid samples, like urine samples, will be buffered directly with the 10× buffer. As a result, the sample is diluted less, allowing our assay to detect the substance more sensitively.

Further, as part of our results, we have considered 40 clinical samples and assessed the performance of the assay. Here, 10 samples were CT +ve, 10 were CT & NG +ve, 10 were only NG +ve and 10 were both CT and NG −ve. The results of our CDNA assay have been demonstrated in FIG. 4. The assay currently provides ˜100% predictive value both for the determination of positive and negative samples.

The assay will be optimized for POC application towards the selective detection of CT and NG directly from the infected media without any purification of DNA.

In order to extract DNA from bacteria, the buffer should rupture the membrane of the bacteria. As a result, the buffer's potency will have a major impact on success.

2. Develop of a functional lateral flow dipstick assay for the selective multiplexed diagnosis of CT and NG. As soon as the proposed CDNA assay has been analytically validated with optimized reagent concentrations, a dipstick assay must be developed for POC deployment of the CT and NG diagnostic assays (Dighe et al., Biosensors and Bioelectronics, (2022), 200:113900).

In this part, we will utilize the optimized concentration of crRNA, linker probe and ssDNAs (now functionalized with biotin and FAM) to develop a functional dipstick assay directly from the clinical samples. We expect to see a band in control line only in presence of target DNAs, whereas bands will be observed in both test and control lines in absence of target DNA. Finally, the assay will be validated for the POC diagnosis of CT and/or NG infection using hospital acquired frozen clinical samples.

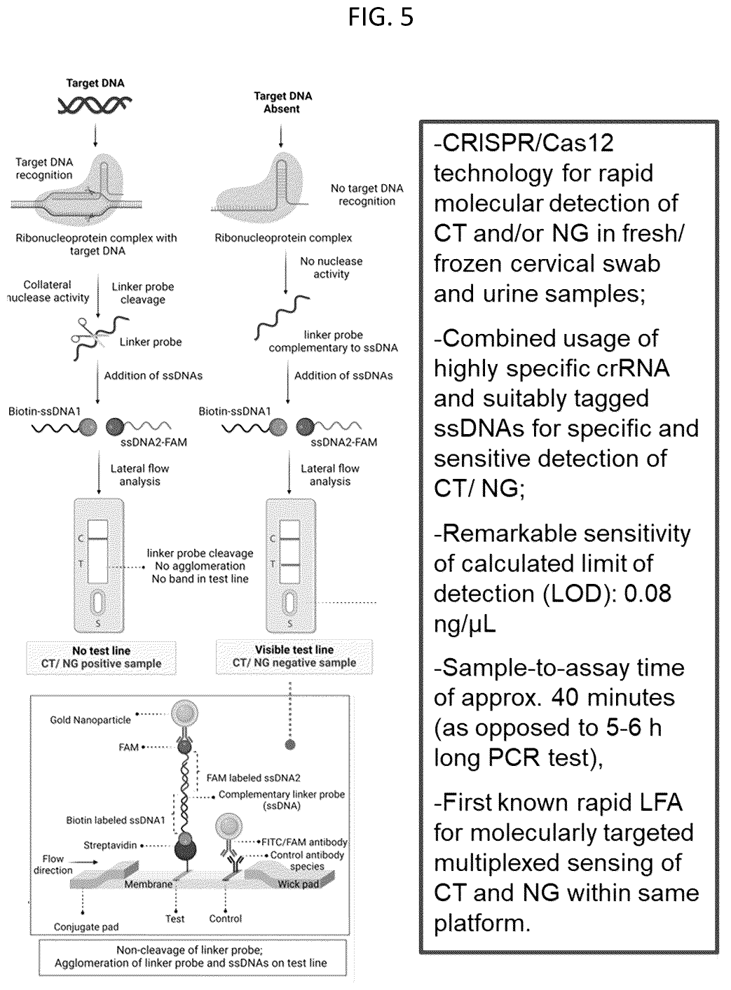

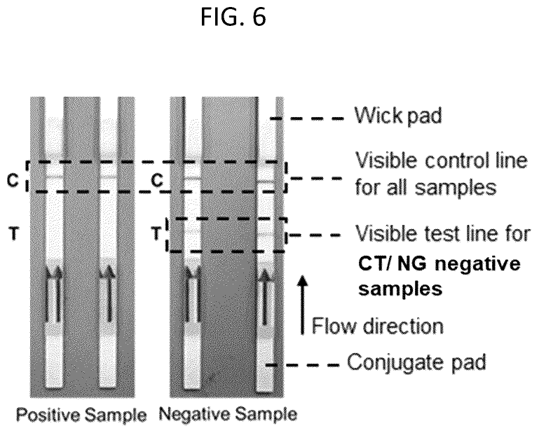

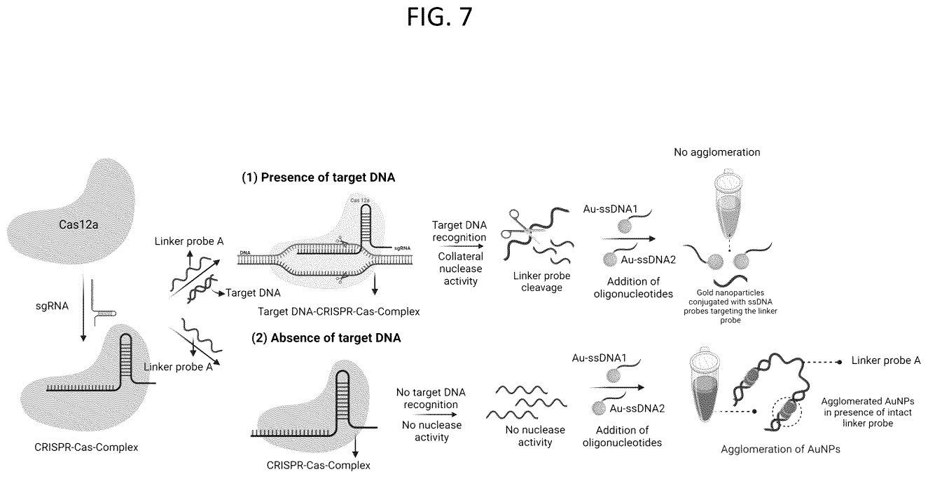

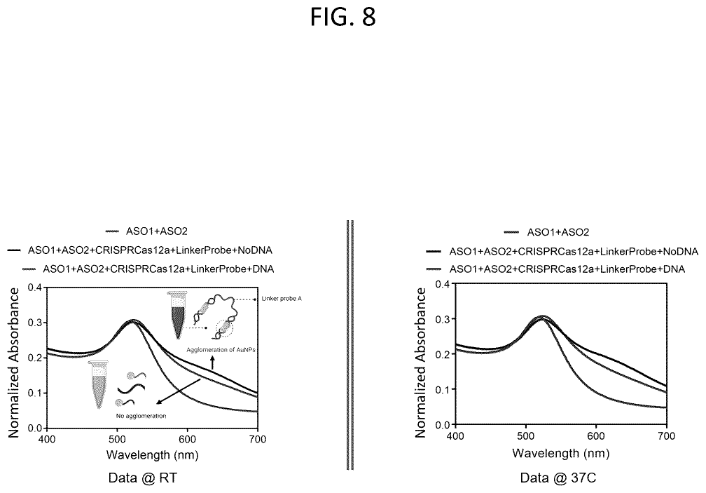

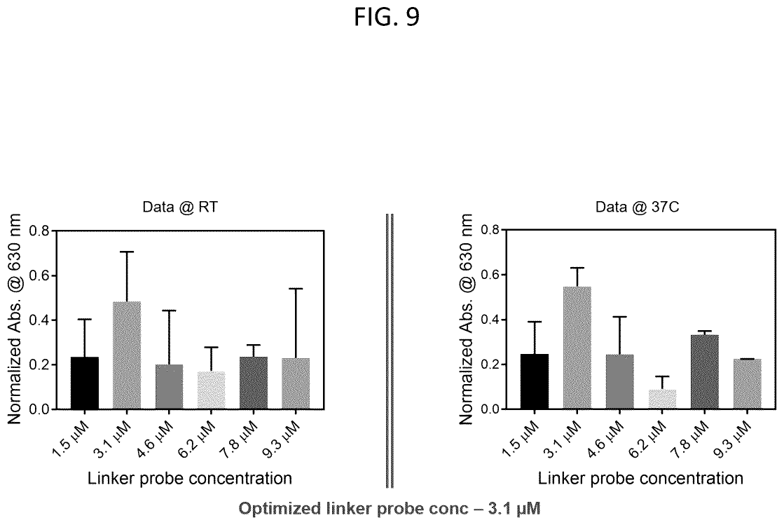

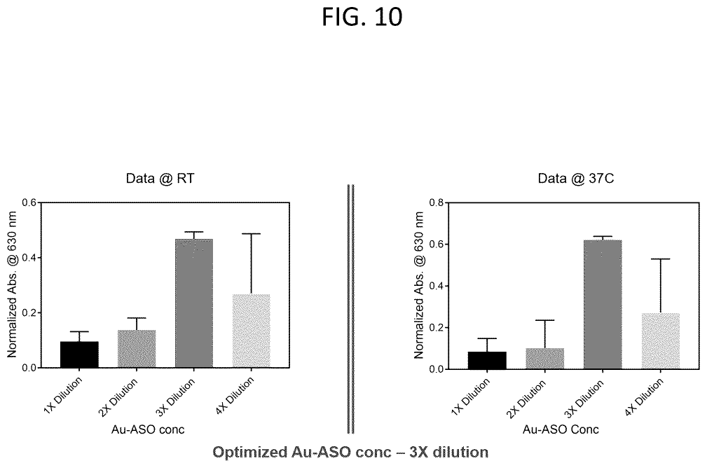

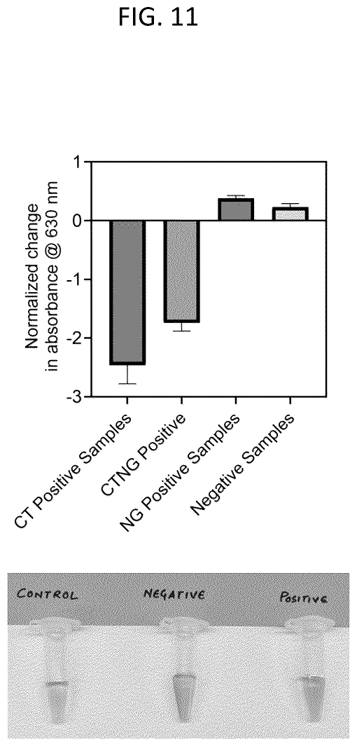

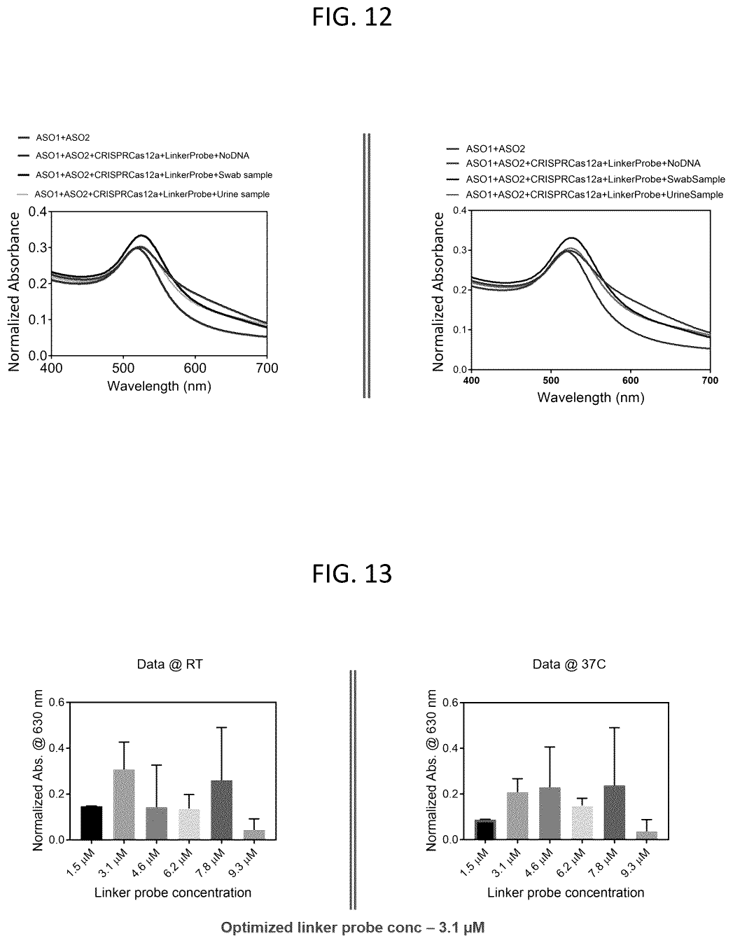

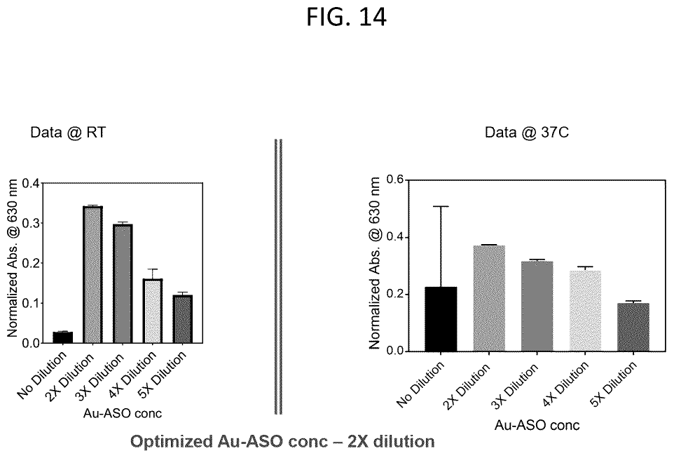

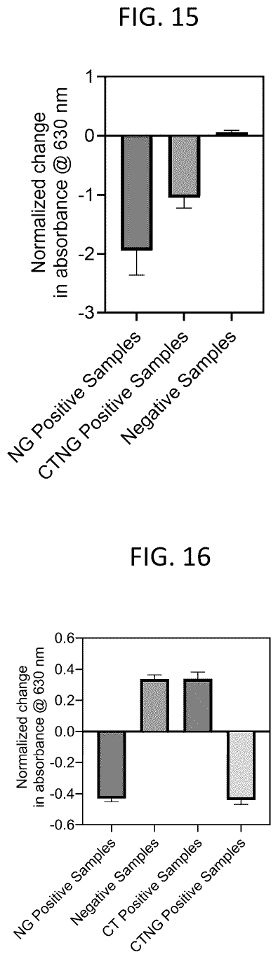

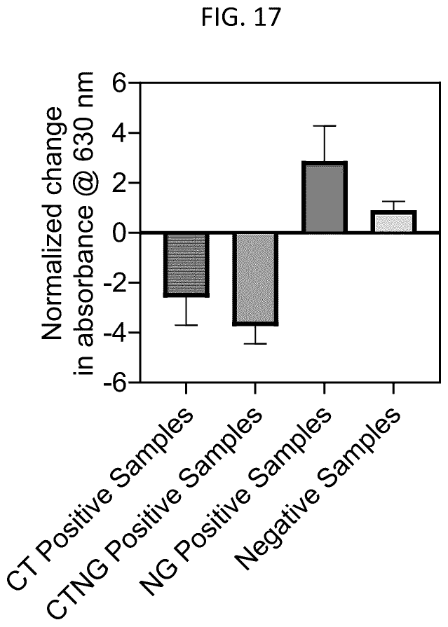

It was demonstrated in part 1 that when target DNA is present, the Cas enzyme can activate its collateral cleavage activity in order to cleave the linker probe, leaving no space for the biotin and FAM tagged ssDNA to bind to it. Hence, we expect to see a band in the control line only in presence of target CT and NG DNA, whereas bands will be observed in both test and control lines in absence of target DNA. (FIG. 5). For feasibility study, we have used one CT +ve, one NG +ve and two negative samples. Briefly, the assay protocol is: 20 μL of nuclease-free water, 3 μL of NEBuffer r2.1 reaction buffer (10×), 3 L (30 nM final concentration) of 300 nM crRNA, and 1 μL (˜30 nM final concentration) of 1 μM EnGen Lba Cas12a (Cpf1) were mixed adequately. This reaction mixture has been incubated for 10 minutes at 25° C. (Zetsche et al., Cell, (2015), 163:759-771; Santana et al., Nature Communications, (2021), 12 (1). https://doi.org/10.1038/s41467-021-27545-5). Subsequently, 2 μL of 30 nM target DNA, 1.5 μL of linker probe and 2.5 μL of nuclease-free water are added to the same suspension. The solution is mixed thoroughly using a pulse-spin microfuge and incubated for 10 minutes. Further, 1 μL of Proteinase K is added, mixed thoroughly and incubated at room temperature for 10 minutes. 10 μL of this resulting solution is then mixed with 100 μL of HybriDetect assay buffer (Zasada et al., BMC infectious Diseases, (2020), 20 (1); Poulton et al., Analytical Biochemistry, (2018), 546:65-71: Zasada et al., Biomedicines, (2022), 10: 2329 Zasada et al., Analytical Biochemistry, (2018), 560:60-66). The dipsticks are then placed with the sample application area and incubated for ˜7-10 minutes in an upright position and the result was read immediately. In the case of the positive sample, only the control line is visible, whereas, for the negative sample both the control and test lines appeared on the dipstick (FIG. 6). We will further study this dipstick-based lateral flow platform with different concentrations of CT and NG gene in the presence and absence of interfering elements. An external positive control, i.e., CT/NG DNA and external negative control, i.e., water will also be provided with the assay.

A dipstick based LFA will be developed and validated both analytically and clinically based. By end of this aim we expect to have optimized the reagent concentrations, DNA concentration and validated the optimal operation of the dipstick against cross-interfering agents.

3. Conduct a blinded clinical study to assess one-step CRISPR-Cas12 based assay against standard of care CTNG assessment. The primary objective of this part will be to evaluate the performance of a CRISPR-Cas12 POC assay directly against a clinical diagnostic gold standard and to assess the impact that a POC CDNA based NALFA device would have on overtreatment, undertreatment and time to treatment for Chlamydia and gonorrhea.

The high potential for transmission of Chlamydia and gonorrhea often results in the overtreatment for these conditions. Rates of overtreatment in emergency departments have been shown to be as great as 86% (Holley et al., The American Journal of Emergency Medicine, (2015). 33:1265-1268; Gatell, J. M., International Journal of STD & AIDS, (2009), 20: (2_suppl), 1-1). That is, 86% of those receiving treatment are negative for the disease. Concurrently, often as much as 50% of those who eventually receive positive results are not provided treatment at the time of testing (Jenkins et al., The Journal of Emergency Medicine, (2013), 44:558-567; Holley et al., The American Journal of Emergency Medicine, (2015), 33:1265-1268). While it is possible to improve treatment rates with intense follow-up procedures, this can be both costly and time intensive. An accurate POC device for diagnosing Chlamydia and gonorrhea infection would greatly improve over- and undertreatment rates as well as decrease the associated cost burden on health care systems. Rates of over- and undertreatment at Carle Foundation Hospital, a medium sized (453-bed) hospital with a service area encompassing rural, urban cluster, and urban populations, will be determined. The impact of a fast, accurate, POC lateral flow device for diagnosing Chlamydia and gonorrhea in this mixed population base will be evaluated.

3a: Create a clinical sample bank.

A bank will be created from samples submitted to the Carle Pathology lab for Chlamydia and gonorrhea testing. In addition to clinical testing, banked samples will be used to bench test POC devices in both an unblended and blinded fashion (see parts 1 and 2).

Samples will be obtained, assigned a research number, and provided to the bank with no identifiable factors. The creation of the bank has been determined to be non-human subjects research as defined by 45 CFR 46.102(d)(f) (see IRB Letter in supporting documents). The bank will consist of both female swabs in stabilizing solution and male urine decanted into the same stabilizing solution. Clinical testing results, as performed on a Roche Molecular Diagnostics Cobas 4800 instrument with the Cobas CT/NG v. 2.0 kit, will be aligned with each sample banked. General sample data including age (by decile), submitting department, and gender will be associated with clinical samples.

The target accrual for the sample bank will be 335 samples for each of (1) female, Chlamydia positive, (2) female, gonorrhea positive, (3) male, Chlamydia positive, and (4) male, gonorrhea positive. Banked samples will be aliquoted into 4 aliquots and stored at −80° C. An additional 400 samples each of (5) female negative and (6) male negative samples will be aliquoted and banked. Samples will be obtained from the Carle Pathology lab beginning in year 1 with accrual goals achieved by end of year 3. Samples tested in 2021 with associated rates of positivity are shown in Table 1. Accrual targets per month for this study are also indicated.

3b: Evaluate Chlamydia and gonorrhea treatment based on current standard of care practices and establish a database to estimate potential impact of an accurate POC device on over- and undertreatment.

TABLE 1
Chlamydia and gonorrhea tests run at Carle Foundation
Hospital during 2021. Monthly averages and accrual
targets for bank creation are included.

| | # Tests | # | % | Monthly | Monthly Accrual |
|---|---|---|---|---|---|
| | Run | Positive | Positive | Avg. | Target |
| Chlamydia | | | | | |
| Female | 11051 | 701 | 6.34% | 58 | 30 |
| Male | 3033 | 309 | 10.19% | 26 | 15 |
| Total | 14084 | 1010 | 7.17% | 84 | 45 |
| Gonorrhea | | | | | |
| Female | 11050 | 305 | 2.76% | 25 | 15 |
| Male | 3034 | 228 | 7.51% | 19 | 12 |
| Total | 14084 | 533 | 3.78% | 44 | 27 |

Rates of over- and undertreatment that result from current standard of care practices will be tracked for samples being tested in the Carle Pathology lab. De-identified treatment rates will be entered into a secure database to provide an estimated impact of reliable, accurate POC testing at Carle Foundation Hospital on over- and undertreatment and time to treatment.

Treatments for Chlamydia and gonorrhea as defined by CDC Treatment Guidelines, 2021 will be determined for samples in the sample bank as well as for all samples during a continuous time period of up to 3 years. See Table 2 for a summary of medications tracked.

TABLE 2
Medication information to be tracked for samples sent
to pathology for chlamydia and gonorrhea testing.

| Chlamydia | Gonorrhea |
|---|---|
| Doxycycline | Ceftriaxone |
| Azythromycin | Cefotaxime |
| Erythromycin | Ceftizoxime |
| Levofloxacin | Ceftriaxone |
| Amoxicillin | Gentamicine |
| | Cefixime |

Specifically, prescriptions provided, and medication delivered will be tracked in a de-identified, retrospective manner uses the hospital's electronic medical records system. Data will be compiled by Carle Foundation Hospital's Stephens Family Clinical Research Institute (SFCRI) data team. Data will be compiled using automated reports. The automated reporting script will be reviewed, refined, and retested by manually reviewing a subset of data during the building of the report. General sample data including age (by decile), submitting department, and gender will be associated with the clinical data. The day of the week that samples are collected will also be recorded. Treatment data will be aligned with banked samples and POC tested samples by an honest broker on the SFCRI team. Treatment for all samples tested in the Carle Pathology lab in years 2 to 4 of the grant will be compiled. Treatment delivery will be defined as “day of” if treatment is provided on the same calendar date that the test sample was collected. Treatments up to 3 days after the day of sample collection will be identified and classified as days +1, +2, +3. Laboratory test results from the Roche Cobas CT/NG v2.0 kit will be considered the “gold standard” in evaluating POC test results. Rates of over- and undertreatment will be based on Roche Cobas results.

Rates of over- and undertreatment of CTNG based on standard of care testing at a mid-sized hospital serving a rural to urban population base. Additionally, for appropriate treatment provided after the day of testing, the time to treatment will be determined.

3c: Performance evaluation of POC device against clinical gold standard assay and impact of POC testing on treatment rates.

300 samples submitted to Carle Pathology lab will be measured serially with a concurrent measurement of an enriched sample set of 300 samples taken from the sample bank. Initial testing will be performed on female swab samples followed by an evaluation of male urine. Samples will be measured on Tuesday to Friday during the study. Samples will not be measured on Mondays due to weekend backlog measurements that take place on this day resulting in multi-day-old samples in the queue. Approximately 30 female swab samples and 10 male urine samples are tested in the lab each Tuesday to Friday. During the female swab study, the first 12 female swab samples arriving in the lab each Tuesday to Friday will be measured with the POC device. These samples will be supplemented with 12 banked samples for a total of 24 samples each measurement day. For the male urine, 8 serial and 12 banked samples will be measured each day. The enriched samples will be randomized to contain 0-12 positive samples each with an average positivity of the enriched subset over the course of the study of 33% positive for Chlamydia and 33% positive for gonorrhea. Enriched sample testing will stop at a total of 300 samples for each of the female swab and male urine studies with the serial testing continuing until 300 of each sample type has been tested. The personnel performing the measurement will be blinded to any existing clinical results while performing the measurement until each study is completed. The numbers of positive and negative samples expected for the study are shown in Table 3 based on historic rates of positivity and enriched sample subset. The female swab study will take approximately 2.5 months to complete and the male urine study 4 months to complete.

During testing, test strips will be evaluated after applying sample at 5, 10, and 15 minutes. Presence or absence of control line, Chlamydia line, and gonorrhea line will be noted at each time point. An image of the test strip will be taken at the recommended read time as determined in parts 1 and 2. Partial lines or deviations from standard positive and negative indications will be noted. Test positivity or negativity for each target marker will be recorded. Samples that do not register a control line in 15 minutes will be retested up to 2 additional times with each retest noted. Serially measured samples and enriched sample set will be evaluated for positive percent agreement (PPA) and negative percent agreement (NPA) both independently and combined to determine variance in fresh versus banked samples against the Roche Cobas CT/NG v2.0 kit results as the clinical gold standard. The test sensitivity and interval range calculated at a 95% confidence will be determined using the combined serial and enriched sample sets.

TABLE 3
Estimated numbers of positives and negatives for POC evaluation
testing using historical positivity rates. Test set will
consist of serially collected samples and an enriched
sample set created from the sample bank.

| | Serial | Enriched | |
|---|---|---|---|
| | (300 samples) | (300 samples) | Total |
| Female swabs | | | |
| Chlamydia | 19 | 100 | 119 |
| Gonorrhea | 8 | 100 | 108 |
| Negative | 273 | 100 | 373 |
| Total | 300 | 300 | 600 |
| Male urine | | | |
| Chlamydia | 31 | 100 | 131 |
| Gonorrhea | 23 | 100 | 123 |
| Negative | 246 | 100 | 346 |
| Total | 300 | 300 | 600 |

Establishment of clinical biosensor parameters-Limit of detection (LoD) and Time of Response (TOR): Benchtop studies will determine optimum conditions for reagents and time of response (TOR) for sample incubation and lateral flow readout. TOR will also be evaluated at 5-minute intervals during clinical sample evaluation. The clinical sample limit of detection (LoD) for the device will be calculated using clinical samples diluted to the analytical LoD using RT-PCR as the comparative reference. The analytical LoD will use defined with known concentrations of target sequence DNA. Clinical samples for LoD determination will be generated by dilution to the analytical LoD, 2×LoD, and 4×LoD. These samples will be measured by the POC device with an n=20. The lowest diluted clinical sample concentration yielding a minimum of 19 positives out of 20 will be the clinical sample LoD.

Statistical Analysis: The target accrual size for the sample bank was determined using a Clopper-Pearson interval calculation with a confidence level of 95%. A bank of 334 positive samples for each disease target and sample type (female swab, male urine) will be sufficient to establish test sensitivity estimated at 95% with a target width of +/−2.5%. Accrual numbers may be adjusted based on results from parts 1 and 2. All banked samples will be aliquoted, allowing use in bench-top development, repeat measurements for reliability and repeatability, and for enrichment of positive sample frequency during clinical evaluation. Performance evaluation of the POC during the clinical evaluation will include agreement with the gold standard using both positive percent agreement (PPA) and negative percent agreement (NPA) calculations. Both PPA and NPA will be calculated using serially tested samples (300 samples from each of female swabs and male urine). PPA and NPA will also be calculated for enriched subset testing and for the combined serial test and enriched test. Additionally, the test sensitivity and specificity for each target and media will be evaluated. Combined serially tested samples and enriched supplemental samples will be used to evaluate sensitivity and specificity. At a 95% confidence level and assuming a normal distribution, for the expected frequency of true positives on the serial and enriched samples, the confidence intervals for different sensitivities are shown in Table 4.

TABLE 4
Calculated confidence intervals for test sensitivity with the expected
numbers of positive samples in the clinical study. Confidence interval
for different test sensitivities is shown for a 95% confidence level
with the estimated number of positive samples from Table 3.

| Test | Female | Female | Male | Male |
|---|---|---|---|---|
| Sensitivity | Chlamydia | Gonorrhea | Chlamydia | Gonorrhea |
| 0.99 | +/−0.018 | +/−0.019 | +/−0.017 | +/−0.018 |
| 0.98 | +/−0.025 | +/−0.026 | +/−0.024 | +/−0.025 |
| 0.95 | +/−0.039 | +/−0.041 | +/−0.037 | +/−0.039 |
| 0.90 | +/−0.054 | +/−0.057 | +/−0.051 | +/−0.053 |
| 0.80 | +/−0.072 | +/−0.075 | +/−0.068 | +/−0.071 |

While the present teachings are described in conjunction with various embodiments, it is not intended that the present teachings be limited to such embodiments. On the contrary, the present teachings encompass various alternatives, modifications, and equivalents, as will be appreciated by those of skill in the art.

1. A method of detecting the presence or absence of nucleic acid from Chlamydia trachomatis and the presence or absence of nucleic acid from Neisseria gonorrhoeae in a sample from a subject, comprising
a. providing the sample from the subject;
b. contacting the sample with i) a complex comprising a CRISPR nuclease having collateral nuclease activity and a crRNA comprising a nucleic acid sequence that is complementary to a nucleic acid sequence of Chlamydia trachomatis; and ii) a first single stranded nucleic acid linker probe;
c. contacting the sample with iii) a complex comprising a CRISPR nuclease having collateral nuclease activity and a crRNA comprising a nucleic acid sequence that is complementary to a nucleic acid sequence of Neisseria gonorrhoeae; and iv) a second single stranded nucleic acid linker probe;
d. incubating the sample with the complexes of parts i) and iii) and single stranded linker probes of parts ii) and iv) for a period of time;
wherein when the nucleic acid sequence of Chlamydia trachomatis is present in the sample, the collateral nuclease activity of the complex of part i) is activated and the first single stranded nucleic acid linker probe or part ii) is cleaved by the nuclease;
wherein when the nucleic acid sequence of Chlamydia trachomatis is not present in the sample, the collateral nuclease activity of the complex of part i) is not activated and the first single stranded nucleic acid linker probe of part ii) is not cleaved;
wherein when the nucleic acid sequence of Neisseria gonorrhoeae is present in the sample, the collateral nuclease activity of the complex of part iii) is activated and the second single stranded nucleic acid linker probe of part iv) is cleaved by the nuclease;
wherein when the nucleic acid sequence of Neisseria gonorrhoeae is not present in the sample, the collateral nuclease activity of the complex of part iii) is not activated and the second single stranded nucleic acid linker probe of part iv) is not cleaved;
e. contacting the sample with a first single stranded nucleic acid that hybridizes to a first nucleic acid sequence of the first single stranded nucleic acid linker probe of part ii) and a second single stranded nucleic acid that hybridizes to a second nucleic acid sequence of the first single stranded nucleic acid linker probe of part ii);
wherein the first and second single stranded nucleic acids hybridize to the first single stranded nucleic acid linker probe of part ii) when the first single stranded nucleic acid linker probe of part ii) is not cleaved, to form a complex comprising the first single stranded nucleic acid linker probe of part ii) and the first and second single stranded nucleic acids;
f. contacting the sample with a third single stranded nucleic acid that hybridizes to a first nucleic acid sequence of the second single stranded nucleic acid linker probe of part iv) and a fourth single stranded nucleic acid that hybridizes to a second nucleic acid sequence of the second single stranded nucleic acid linker probe of part iv);
wherein the third and fourth single stranded nucleic acids hybridize to the second single stranded nucleic acid linker probe of part iv) when the second single stranded nucleic acid linker probe of part iv) is not cleaved, to form a complex comprising the second single stranded nucleic acid linker probe of part iv) and the third and fourth single stranded nucleic acids;
g. detecting the presence or absence of the complex comprising the first single stranded nucleic acid linker probe of part ii) hybridized to the first and second single stranded nucleic acids, thereby detecting the presence or absence of nucleic acid from Chlamydia trachomatis in the sample; and
h. detecting the presence or absence of the complex comprising the second single stranded nucleic acid linker probe of part iv) hybridized to the third and fourth single stranded nucleic acids, thereby detecting the presence or absence of nucleic acid from Neisseria gonorrhoeae in the sample.

2. The method of claim 1, wherein the sample has been treated with an agent to release the nucleic acids from Chlamydia trachomatis and Neisseria gonorrhoeae, wherein the agent lyses the cells.

3. (canceled)

4. (canceled)

5. The method of claim 1, wherein the crRNA is complementary to a nucleic acid sequence of a target gene, wherein the target gene of Chlamydia trachomatis is cryptic plasmid ORF6 segment sequence and the target gene of Neisseria gonorrhoeae is a major outer membrane protein porB.

6. The method of claim 1, wherein the first single stranded linker probe comprises a nucleic acid sequence of the target gene of Chlamydia trachomatis and the second single stranded linker probe comprises a nucleic acid sequence of the target gene of Neisseria gonorrhoeae.

7. The method of claim 6, wherein the crRNA that is complementary to cryptic plasmid ORF6 segment sequence comprises SEQ ID NO:10, wherein the crRNA that is complementary to major outer membrane protein porB sequence comprises SEQ ID NO:24.

8.-10. (canceled)

11. The method of claim 1, wherein the CRISPR nuclease comprises a Cas12a nuclease.

12. The method of claim 1, wherein the single stranded nucleic acid linker probe of part ii) comprises SEQ ID NO:5, wherein the single stranded nucleic acid linker probe of part iv) comprises SEQ ID NO:17, wherein the first single stranded nucleic acid comprises SEQ ID NO:3, wherein the second single stranded nucleic acid comprises SEQ ID NO:4, wherein the third single stranded nucleic acid comprises SEQ ID NO:15, wherein the fourth single stranded nucleic acid comprises SEQ ID NO:16.

13.-17. (canceled)

18. The method of claim 1, wherein the first, second, third and/or fourth single stranded nucleic acids are bound to one or more labels, wherein the label is selected from any one of a fluorescent label, radiolabel, streptavidin, an antibody, 6-carboxyfluorescein (FAM), biotin, a thiol group located on the 5′ or 3′ end of the single stranded nucleic acid, a nanoparticle, and combinations thereof.

19. (canceled)

20. (canceled)

21. The method of claim 1, wherein the first single stranded nucleic acid is bound to a gold nanoparticle and the second single stranded nucleic acid is bound to a gold nanoparticle, wherein the third single stranded nucleic acid is bound to a gold nanoparticle and the fourth single stranded nucleic acid is bound to a gold nanoparticle.

22. (canceled)

23. The method of claim 20, wherein a single gold nanoparticle is bound to multiple single stranded nucleic acids.

24. The method of claim 1, wherein the complex comprising the single stranded nucleic acid linker probes of parts ii) and/or iv) hybridized to the single stranded nucleic acids form agglomerates, wherein a sample comprising the agglomerates has a different absorbance measurement than a sample that lacks agglomerates.

25. (canceled)

26. (canceled)

27. The method of claim 18, wherein the first single stranded nucleic acid is bound to 6-carboxyfluorescein (FAM) and the second single stranded nucleic acid is bound to biotin or wherein the second single stranded nucleic acid is bound to 6-carboxyfluorescein (FAM) and the first single stranded nucleic acid is bound to biotin, wherein the third single stranded nucleic acid is bound to 6-carboxyfluorescein (FAM) and the fourth single stranded nucleic acid is bound to biotin or wherein the fourth single stranded nucleic acid is bound to 6-carboxyfluorescein (FAM) and the third single stranded nucleic acid is bound to biotin.

28. (canceled)

29. The method of claim 1, further comprising loading the sample comprising the single stranded linker probes and the single stranded nucleic acids onto a lateral flow assay to detect the presence or absence of the complex comprising the single stranded nucleic acid linker probes hybridized to the single stranded nucleic acids.

30. (canceled)

31. The method of claim 29, wherein the lateral flow assay comprises streptavidin in a test strip.

32. The method of claim 18, wherein an antibody is added to the sample that binds one or more labels on the single stranded nucleic acid, wherein the antibody is conjugated to a detectable label.

33.-35. (canceled)

36. The method of claim 1, wherein the sample from the subject comprises a single sample, wherein detecting the presence or absence of nucleic acid from Chlamydia trachomatis and Neisseria gonorrhoeae is performed on a single sample.

37. The method of claim 1, wherein the sample from the subject comprises a first sample for detecting the presence or absence of nucleic acid from Chlamydia trachomatis and a second sample for detecting the presence or absence of nucleic acid from Neisseria gonorrhoeae.

38. (canceled)

39. A method of detecting the presence or absence of nucleic acid from Chlamydia trachomatis in a sample from a subject, comprising
a. providing the sample from the subject;
b. contacting the sample with i) a complex comprising a CRISPR nuclease having collateral nuclease activity and a crRNA comprising a nucleic acid sequence that is complementary to a nucleic acid sequence of Chlamydia trachomatis; and ii) a single stranded nucleic acid linker probe;
c. incubating the sample with the complex of part i) and single stranded linker probe of part ii) for a period of time;
wherein when the nucleic acid sequence of Chlamydia trachomatis is present in the sample, the collateral nuclease activity of the complex of part i) is activated and the single stranded nucleic acid linker probe or part ii) is cleaved by the nuclease;
wherein when the nucleic acid sequence of Chlamydia trachomatis is not present in the sample, the collateral nuclease activity of the complex of part i) is not activated and the single stranded nucleic acid linker probe of part ii) is not cleaved;
d. contacting the sample with a first single stranded nucleic acid that hybridizes to a first nucleic acid sequence of the single stranded nucleic acid linker probe of part ii) and a second single stranded nucleic acid that hybridizes to a second nucleic acid sequence of the single stranded nucleic acid linker probe of part ii);
wherein the first and second single stranded nucleic acids hybridize to the single stranded nucleic acid linker probe of part ii) when the single stranded nucleic acid linker probe of part ii) is not cleaved, to form a complex comprising the single stranded nucleic acid linker probe of part ii) and the first and second single stranded nucleic acids;
e. detecting the presence or absence of the complex comprising the single stranded nucleic acid linker probe of part ii) hybridized to the first and second single stranded nucleic acids, thereby detecting the presence or absence of nucleic acid from Chlamydia trachomatis in the sample.

40.-68. (canceled)

69. A method of detecting the presence or absence of nucleic acid from Neisseria gonorrhoeae in a sample from a subject, comprising
a. providing the sample from the subject;
b. contacting the sample with i) a complex comprising a CRISPR nuclease having collateral nuclease activity and a crRNA comprising a nucleic acid sequence that is complementary to a nucleic acid sequence of Neisseria gonorrhoeae; and ii) a single stranded nucleic acid linker probe;
c. incubating the sample with the complexes of part i) and single stranded linker probe of part ii) for a period of time;
wherein when the nucleic acid sequence of Neisseria gonorrhoeae is present in the sample, the collateral nuclease activity of the complex of part i) is activated and the single stranded nucleic acid linker probe of part ii) is cleaved by the nuclease;
wherein when the nucleic acid sequence of Neisseria gonorrhoeae is not present in the sample, the collateral nuclease activity of the complex of part i) is not activated and the single stranded nucleic acid linker probe of part ii) is not cleaved;
d. contacting the sample with a first single stranded nucleic acid that hybridizes to a first nucleic acid sequence of the single stranded nucleic acid linker probe of part ii) and a second single stranded nucleic acid that hybridizes to a second nucleic acid sequence of the single stranded nucleic acid linker probe of part ii);
wherein the first and second single stranded nucleic acids hybridize to the single stranded nucleic acid linker probe of part ii) when the single stranded nucleic acid linker probe of part ii) is not cleaved, to form a complex comprising the single stranded nucleic acid linker probe of part ii) and the first and second single stranded nucleic acids;
e. detecting the presence or absence of the complex comprising the stranded nucleic acid linker probe of part ii) hybridized to the first and second single stranded nucleic acids, thereby detecting the presence or absence of nucleic acid from Neisseria gonorrhoeae in the sample.

70.-99. (canceled)

100. The method of claim 1, further comprising administering to the subject an effective amount of one or more agents to treat an infection caused by Chlamydia trachomatis and/or Neisseria gonorrhoeae.

101. (canceled)

102. (canceled)


SEQUENCE LISTING (36 sequences)

SEQ ID NO: 1 - protein, 147 aa, Chlamydia trachomatis
FKKTLPSIELFSATLNSEESQSLDQLFLSESQNYSDEEFYQEDILAVKLLTGQIKSIQKQHVLLLGEKIYNARKILSKDHFSSTTFSSWIELVFRTKSSAYNALAYYELFINLPNQTLQKEFQSIPYKSAYILAARKGDLKTKVDVI

SEQ ID NO: 2 - DNA, 441 bp, Chlamydia trachomatis
  source 1..441: mol_type = other DNA
tttaagaagacgcttccttccattgaactattctcagcaactttgaattctgaggaaagtcagagtttggatcaattatttttatcagagtcccaaaactattcggatgaagaattttatcaagaagacatcctagcggtaaaactgcttactggtcagataaaatccatacagaagcaacacgtacttcttttaggagaaaaaatctataatgctagaaaaatcctgagtaaggatcacttctcctcaacaactttttcatcttggatagagttagtttttagaactaagtcttctgcttacaatgctcttgcatattacgagctttttataaacctccccaaccaaactctacaaaaagagtttcaatcgatcccctataaatccgcatatattttggccgctagaaaaggcgatttaaaaaccaaggtcgatgtgata

SEQ ID NO: 3 - DNA, 20 bp, synthetic construct
  source 1..20: mol_type = other DNA
tggttggggaggtttataaa

SEQ ID NO: 4 - DNA, 20 bp, synthetic construct
  source 1..20: mol_type = other DNA
ggatttataggggatcgatt

SEQ ID NO: 5 - DNA, 65 bp, synthetic construct
  source 1..65: mol_type = other DNA
tttttataaacctccccaaccaaactctacaaaaagagtttcaatcgatcccctataaatccgca

SEQ ID NO: 6 - DNA, 28 bp, synthetic construct
  source 1..28: mol_type = other DNA
ttccattgaactattctcagcaactttg

SEQ ID NO: 7 - DNA, 28 bp, synthetic construct
  source 1..28: mol_type = other DNA
ttctcctaaaagaagtacgtgttgcttc

SEQ ID NO: 8 - DNA, 28 bp, synthetic construct
  source 1..28: mol_type = other DNA
ttcttttaggagaaaaaatctataatgc

SEQ ID NO: 9 - DNA, 29 bp, synthetic construct
  source 1..29: mol_type = other DNA
tttctcctaaaagaagtacgtgttgcttc

SEQ ID NO: 10 - RNA, 41 nt, synthetic construct
  source 1..41: mol_type = other RNA
taatttctactaagtgtagattcctaaaagaagtacgtgtt

SEQ ID NO: 11 - DNA, 24 bp, synthetic construct
  source 1..24: mol_type = other DNA
agcggtaaaactgcttactggtca

SEQ ID NO: 12 - DNA, 20 bp, synthetic construct
  source 1..20: mol_type = other DNA
tcgccttttctagcggccaa

SEQ ID NO: 13 - protein, 350 aa, Neisseria gonorrhoeae
MKKSLIALTLAALPVAATADVTLYGAIKAGVQTYRSVEHTDGKVSKVETGSEIADFGSKIGFKGQEDLGNGLKAVWQLEQGASVAGTNTGWGNKQSFVGLKGGFGTIRAGSLNSPLKNTDDNVNAWESGKFTGNVLEISGMAKREHRYLSVRYDSPEFAGFSGSVQYAPKDNSGSNGESYHVGLNYQNSGFFAQYAGLFQRYGEGTKKIEYEHQVYSIPSLFVEKLQVHRLVGGYDNNALYVSVAAQQQDAKLYQNQLVRDNSHNSQTEVAATVAYRFGNVTPRVSYAHGFKGTVDSADHDNTYDQVVVGAEYDFSKRTSALVSAGWLQEGKGADKIVSTASAVVLRHKF

SEQ ID NO: 14 - DNA, 1050 bp, Neisseria gonorrhoeae
  source 1..1050: mol_type = other DNA
atgaaaaaatccctgattgccctgactttggcagcccttcctgttgcggcaacggccgatgtcaccctgtacggtgccatcaaagccggcgtacaaacttaccgttctgtagaacatacagacggcaaggtaagtaaagtggaaaccggcagcgaaatcgccgacttcggttcaaaaatcggcttcaaaggccaagaagacctcggcaacggtctgaaggccgtttggcagttggaacaaggtgcctccgtcgccggcactaacaccggctggggcaacaaacaatccttcgtcggcttgaagggcggcttcggtaccatccgcgccggtagcctgaacagccccctgaaaaacaccgacgacaacgtcaatgcttgggaatccggcaaatttaccggcaatgtgctggaaatcagcggaatggccaaacgggaacaccgctacctgtccgtacgctacgattctcccgaatttgccggcttcagcggcagcgtacaatacgcacctaaagacaattcaggctcaaacggcgaatcttaccacgttggcttgaactaccaaaacagcggcttcttcgcacaatacgccggcttgttccaaagatacggcgaaggcactaaaaaaatcgaatacgaacatcaagtttatagtatccccagcctgtttgttgaaaaactgcaagttcaccgtttggtaggcggttacgacaataatgccctgtacgtctccgtagccgcacaacaacaagatgccaaattgtatcaaaatcaattagtgcgtgataattcgcacaactctcaaaccgaagttgccgctaccgtggcataccgtttcggcaacgtaacgccccgcgtttcttacgcccacggcttcaaaggcactgttgatagtgcagaccacgacaatacttatgaccaagtggttgtcggcgcggaatacgacttctccaaacgcacttctgccttggtttctgccggctggttgcaagaaggcaaaggcgcagacaaaatcgtatcgactgccagcgccgtcgttctgcgccacaaattc

SEQ ID NO: 15 - DNA, 20 bp, synthetic construct
  source 1..20: mol_type = other DNA
ttgtctttaggtgcgtattg

SEQ ID NO: 16 - DNA, 20 bp, synthetic construct
  source 1..20: mol_type = other DNA
tggtaagattcgccgtttga

SEQ ID NO: 17 - DNA, 55 bp, synthetic construct
  source 1..55: mol_type = other DNA
gcgtacaatacgcacctaaagacaattcaggctcaaacggcgaatcttaccacgt

SEQ ID NO: 18 - DNA, 28 bp, synthetic construct
  source 1..28: mol_type = other DNA
ttctgtagaacatacagacggcaaggta

SEQ ID NO: 19 - DNA, 28 bp, synthetic construct
  source 1..28: mol_type = other DNA
ttccactttacttaccttgccgtctgta

SEQ ID NO: 20 - DNA, 28 bp, synthetic construct
  source 1..28: mol_type = other DNA
ttcgctgccggtttccactttacttacc

SEQ ID NO: 21 - DNA, 28 bp, synthetic construct
  source 1..28: mol_type = other DNA
ttcggttcaaaaatcggcttcaaaggcc

SEQ ID NO: 22 - DNA, 28 bp, synthetic construct
  source 1..28: mol_type = other DNA
ttcaaaaatcggcttcaaaggccaagaa

SEQ ID NO: 23 - DNA, 29 bp, synthetic construct
  source 1..29: mol_type = other DNA
tttccactttacttaccttgccgtctgta

SEQ ID NO: 24 - RNA, 41 nt, synthetic construct
  source 1..41: mol_type = other RNA
taatttctactaagtgtagatcactttacttaccttgccgt

SEQ ID NO: 25 - DNA, 20 bp, synthetic construct
  source 1..20: mol_type = other DNA
acggcgaatcttaccacgtt

SEQ ID NO: 26 - DNA, 20 bp, synthetic construct
  source 1..20: mol_type = other DNA
tggcatcttgttgttgtgcg

SEQ ID NO: 27 - RNA, 20 nt, Chlamydia trachomatis
  source 1..20: mol_type = other RNA
ccaagagcagcgcctacaac

SEQ ID NO: 28 - RNA, 20 nt, Chlamydia trachomatis
  source 1..20: mol_type = other RNA
tcaacctgcccaaccagacc

SEQ ID NO: 29 - RNA, 20 nt, Neisseria gonorrhoeae
  source 1..20: mol_type = other RNA
cggcgaaggcaccaaaaaaa

SEQ ID NO: 30 - RNA, 20 nt, Neisseria gonorrhoeae
  source 1..20: mol_type = other RNA
accaagtctacagcatcccg

SEQ ID NO: 31 - DNA, 20 bp, Chlamydia trachomatis
  source 1..20: mol_type = other DNA
gttgtaggcgctgctcttgg

SEQ ID NO: 32 - DNA, 20 bp, Chlamydia trachomatis
  source 1..20: mol_type = other DNA
ggtctggttgggcaggttga

SEQ ID NO: 33 - DNA, 20 bp, Neisseria gonorrhoeae
  source 1..20: mol_type = other DNA
tttttttggtgccttcgccg

SEQ ID NO: 34 - DNA, 20 bp, Neisseria gonorrhoeae
  source 1..20: mol_type = other DNA
cgggatgctgtagacttggt

SEQ ID NO: 35 - protein, 1228 aa, Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 36 - DNA, 3684 bp, synthetic construct
  source 1..3684: mol_type = other DNA
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac